(19) United States
(12) Patent Application Publication
(10) Pub. No.: US 2003/0157061 A1
(43) Pub. Date: Aug. 21, 2003

(54) Combinations of a cyclooxygenase-2 selective inhibitor and a TNFalpha antagonist and therapeutic uses therefor

(75) Inventor: Dennis A. Bennett, Wildwood, MO (US)

Correspondence Address: Charles E. Dunlap, Keenan Building, Third Floor, 1330 Lady Street, Columbia, SC, 29201, US

(73) Assignee: Pharmacia Corporation, St. Louis, MO (US)

(21) Appl. No.: 10/310,454

(22) Filed: Dec. 5, 2002

Related U.S. Application Data
(60) Provisional application No. 60/337,802, filed on Dec. 5, 2001.

(51) Int. Cl. A61K 39/395; A61K 38/20; A61K 31/56; A61K 31/415; A61K 31/203; A61K 31/557

(52) U.S. Cl. 424/85.2; 424/145.1; 514/179; 514/406; 514/559; 514/573

(57) ABSTRACT

A method for the prevention, treatment, or inhibition of pain, inflammation, or inflammation-related disorder and for the prevention, treatment, or inhibition of a cardiovascular disease or disorder in a subject that is in need of such prevention, treatment or inhibition, involves the administration to the subject of a cyclooxygenase-2 selective inhibitor or prodrug thereof and a TNFα antagonist. A method can also involve the treatment, prevention, or inhibition of cancer in a subject in need of such treatment, prevention, or inhibition, by administering to the subject a cyclooxygenase-2 selective inhibitor or prodrug thereof and a TNFα antagonist which is selected from the group consisting of a compound that affects the synthesis of TNFα, a compound that inhibits the binding of TNFα with a receptor specific for TNFα, and a compound that interferes with intracellular signaling triggered by TNFα binding with a receptor. Compositions, pharmaceutical compositions and kits that can be used with the methods are also described.

### BACKGROUND OF THE INVENTION

(1) Field of the Invention

The present invention relates to combinations of a cyclooxygenase-2 selective inhibitor and a TNFα antagonist and therapeutic uses for such combinations, and more particularly to combinations of a cyclooxygenase-2 selective inhibitor and a TNFα antagonist and methods of using such combinations for the treatment, prevention or inhibition of pain, inflammation, or inflammation related disorder, or for the treatment, prevention or inhibition of cardiovascular disease or disorder, or for the treatment, prevention or inhibition of cancer.

(2) Description of the Related Art

The ability to treat physical disorders and diseases more effectively has been greatly strengthened by the discovery of drugs that have narrow biological specificity, and which exhibit fewer undesired or unpredicted side effects than drugs having broader activity spectra. The administration of certain combinations of these drugs has provided added benefits including increased efficacy and minimization of undesirable side effects. Two notable examples among the many compounds that recently have been found to be pharmacologically useful are the cyclooxygenase-2 selective inhibitors and the tumor necrosis factor (TNF) antagonists, in particular, those TNF antagonists that inhibit the activity of tumor necrosis factor alpha (TNFα).

Cyclooxygenase-2 selective inhibitors act to regulate the production of certain prostaglandins, which are known to be important mediators of inflammation, as well as to regulate other significant, non-inflammation-related, functions. Regulation of the production and activity of prostaglandins has been a common target of antiinflammatory drug discovery activities. However, common non-steroidal antiinflammatory drugs (NSAIDs) that are active in reducing the prostaglandin-induced pain and swelling associated with the inflammation process also have an effect, sometimes adverse, upon other prostaglandin-regulated processes not associated with the inflammation process. The use of high doses of many common NSAIDs can produce severe side effects that limit their therapeutic potential.

The mechanism ascribed to many of the common NSAIDs is the modulation of prostaglandin synthesis by inhibition of cyclooxygenases that catalyze the transformation of arachidonic acid—the first step in the prostaglandin synthesis pathway. It has recently been discovered that two cyclooxygenases are involved in this transformation. These enzymes have been termed cyclooxygenase-1 (Cox-1) and cyclooxygenase-2 (Cox-2). See, Needleman, P. et al., J. Rheumatol., 24, Suppl.49:6-8 (1997). See, Fu, J. Y., et al., J. Biol. Chem., 265(28):16737-40 (1990).

Cox-1 has been shown to be a constitutively produced enzyme that is involved in many of the non-inflammatory regulatory functions associated with prostaglandins. Cox-2, on the other hand, is an inducible enzyme having significant involvement in the inflammatory process. Inflammation causes the induction of Cox-2, leading to the release of prostanoids, which sensitize peripheral nociceptor terminals and produce localized pain hypersensitivity. See, e.g., Samad, T. A. et al., Nature, 410(6827):471-5 (2001). Many of the common NSAIDs are now known to be inhibitors of both Cox-1 and Cox-2. Accordingly, when administered in sufficiently high levels, these NSAIDs affect not only the inflammatory consequences of Cox-2 activity, but also the beneficial activities of Cox-1.

Recently, compounds that selectively inhibit cyclooxygenase-2 to a greater extent than the activity of Cox-1 have been discovered. The new Cox-2-selective inhibitors are believed to offer advantages that include the capacity to prevent or reduce inflammation while avoiding harmful side effects associated with the inhibition of Cox-1. Thus, cyclooxygenase-2-selective inhibitors have shown great promise for use in therapies—especially those which require extended administration, such as for pain and inflammation control for arthritis. Additional information on the identification of cyclooxygenase-2-selective inhibitors can be found in references such as: Buttgereit, F. et al., Am. J. Med., 110(3 Suppl. 1):13-9 (2001); Osiri, M. et al, Arthritis Care Res., 12(5):351-62 (1999); Buttar, N. S. et al., Mayo Clin. Proc., 75(10):1027-38 (2000); Wollheim, F. A., Current Opin. Rheumatol., 13:193-201 (2001); U.S. Pat. Nos. 5,434,178 (1,3,5-trisubstituted pyrazole compounds); 5,476,944 (derivatives of cyclic phenolic thioethers); 5,643,933 (substituted sulfonylphenylheterocycles); 5,859,257 (isoxazole compounds); 5,932,598 (prodrugs of benzenesulfonamide-containing Cox-2 inhibitors); 6,156,781 (substituted pyrazolyl benzenesulfonamides); and 6,110,960 (for dihydrobenzopyran and related compounds).

The identity, efficacy and side effects of new cyclooxygenase-2-selective inhibitors for the treatment of inflammation have been reported. References include: Hillson, J. L. et al., Expert Opin. Pharmacother., 1(5):1053-66 (2000), (for rofecoxib, Vioxx®, Merck & Co., Inc.); Everts, B. et al., Clin. Rheumatol., 19(5):331-43 (2000), (for celecoxib, Celebrex®, Pharmacia Corporation, and rofecoxib); Jamali, F., J. Pharm. Pharm. Sci., 4(1):1-6 (2001), (for celecoxib); U.S. Pat. Nos. 5,521,207 and 5,760,068 (for substituted pyrazolyl benzenesulfonamides); Davies, N. M. et al., Clinical Genetics, Abstr. at http://www.mmhc.com/cg/articles/CG0006/davies.html (for meloxicam, celecoxib, valdecoxib, parecoxib, deracoxib, and rofecoxib); http://www.celebrex.com (for celecoxib); http://www.docguide.com/dg.nsf/PrintPrint/F1F8DDD2D8B0094085256 98F00742187, May 9, 2001 (for etoricoxib, MK-663, Merck & Co., Inc.); Saag, K. et al., Arch. Fam. Med., 9(10):1124-34 (2000), (for rofecoxib); and in International Patent Publication No. WO 00/24719 (for ABT 963, Abbott Laboratories).

Cox-2 inhibitors have also been described for the treatment of cancer (WO98/16227) and for the treatment of tumors (EP 927,555). Celecoxib®, a specific inhibitor of cox-2, exerted a potent inhibition of fibroblast growth factor-induced corneal angiogenesis in rats. (Masferrer et al., Proc. Am. Assoc. Cancer Research 1999, 40: 396). WO 98/41511 describes 5-(4-sulphonyl-phenyl)-pyridazinone derivatives used for treating cancer. WO 98/41516 describes (methylsulphonyl)phenyl-2-(5H)-furanone derivatives that can be used in the treatment of cancer. Kalgutkar, A. S. et al., Curr. Drug Targets, 2(1):79-106 (2001) suggest that cox-2 selective inhibitors could be used to prevent or treat cancer by affecting tumor viability, growth, and metastasis.

Additionally, various combination therapies using Cox-2 inhibitors with other selected combination regimens for the treatment of cancer has also been reported. See e.g., FR 27 71 005 (compositions containing a cyclooxygenase-2 inhibitor and N-methyl-d-aspartate (NMDA) antagonist used to treat cancer and other diseases); WO 99/18960 (combination comprising a cyclooxygenase-2 inhibitor and an induced nitric-oxide synthase inhibitor (iNOS inhibitor) that can be used to treat colorectal and breast cancer); WO 99/13799 (combination of a cyclooxygenase-2 inhibitor and an opioid analgesic); WO 97/36497 (combination comprising a cyclooxygenase-2 inhibitor and a 5-lipoxygenase inhibitor useful in treating cancer); WO 97/29776 (composition comprising a cyclooxygenase-2 inhibitor in combination with a leukotriene B4 receptor antagonist and an immunosuppressive drug); WO 97/29775 (use of a cyclooxygenase-2 inhibitor in combination with a leukotriene A4 hydrolase inhibitor and an immunosuppressive drug); WO 97/29774 (combination of a cyclooxygenase-2 inhibitor and prostagladin or antiulcer agent useful in treating cancer); WO 97/11701 (combination comprising of a cyclooxygenase-2 inhibitor and a leukotriene B receptor antagonist useful in treating colorectal cancer); WO 96/41645 (combination comprising a cyclooxygenase-2 inhibitor and leukotriene A hydrolase inhibitor); WO 96/03385 (3,4,-Di substituted pyrazole compounds given alone or in combination with NSAIDs, steroids, 5-LO inhibitors, LTB4 antagonists, or LTA4 hydrolase inhibitors for the treatment of cancer); WO 98/47890 (substituted benzopyran derivatives that may be used alone or in combination with other active principles); WO 00/38730 (method of using cyclooxygenase-2 inhibitor and one or more antineoplastic agents as a combination therapy in the treatment of neoplasia); Mann, M. et al., Gastroenterology, 120(7):1713-1719 (2001) (combination treatment with Cox-2 inhibitors and HER-2/neu inhibitors reduced colorectal carcinoma growth).

In (U.S. patent application Ser. No. 09/470,951, filed Dec. 22, 1999), the use of an antiangiogenesis agent in combination with a second material, which could be a cyclooxygenase-2 inhibitor, is disclosed for the treatment of neoplasia disorders. The use of a cyclooxygenase-2 inhibitor with one or more antineoplastic agents is disclosed as a combination therapy for neoplasia in (U.S. patent application Ser. No. 09/857,873, filed Dec. 22, 1999).

Other reports have indicated that Cox-2 selective inhibitors have cardiovascular applications. For example, Saito, T. et al., in Biochem. Biophys. Res. Comm., 273:772-775 (2000), reported that the inhibition of a Cox-2 selective inhibitor improves cardiac function in myocardial infarction. Ridker, P. M. et al., in The New England J. of Med., 336(14):973-979 (1997), raised the possibility that anti-inflammatory agents may have clinical benefits in preventing cardiovascular disease. In addition, Cox-2 selective inhibitors have been proposed for therapeutic use in cardiovascular disease when combined with modulation of inducible nitric oxide synthase (See, Baker, C. S. R. et al., Arterioscler. Thromb. Vasc. Biol., 19:646-655 (1999)), and with HMG-CoA reductase inhibitor (U.S. Pat. No. 6,245,797).

Tumor necrosis factor is a key proinflammatory cytokine released by a number of cell types, particularly activated macrophages and monocytes. Two forms of TNF are released—TNF-alpha (TNFα) and TNF-beta. TNFα is a soluble homotrimer of 17 kD protein subunits (Smith et al., J. Biol. Chem. 262:6951-6954 (1987). A membrane-bound 26 kD precursor form of TNF also exists as a pro-protein and must be cleaved to produce the 17 kD TNF building unit. (Kriegler, et al., Cell, 53:45-53 (1988).

TNF participates in the killing of bacteria and other microorganisms by inducing a variety of cells such as B-cells, T-cells, endothelial cells, and neutrophils to secrete pro-inflammatory factors capable of enhancing the inflammatory response. If the inflammation-inducing stimulus persists for too long, the body's own cells will be damaged. Therefore, the production and secretion of TNF must be closely regulated to avoid harmful results.

Excessive or unregulated TNF production has been implicated in mediating or exacerbating a number of diseases including rheumatoid arthritis, rheumatoid spondylitis, osteoarthritis, gouty arthritis, sepsis, septic shock, endotoxic shock, toxic shock syndrome, adult respiration distress syndrome, cerebral malaria, chronic pulmonary inflammatory disease, silicosis, bone resorption diseases, reperfusion injury, graft vs. host reaction, allograft reaction, fever and myalgias due to infection, cachexia, immune deficiency syndrome (AIDS), AIDS related complex (ARC), keloid formation, scar tissue formation, Crohn's disease, ulcerative colitis, and pyresis.

Several different classes of compounds have been discovered that affect TNF activity. For example, some compounds regulate the synthesis of TNF, while others affect the maturation of TNF, and yet others inhibit the binding of TNF with receptors on the surface of target cells. In addition, a few compounds have been found to affect the intracellular signaling that is triggered by the binding of TNF to a receptor.

The use of TNFα specific antibodies to bind with TNFα and thereby to interfere with its binding to its normal cell receptors has been reported by several groups. For example, Cerami, et al., in EP 0212489, described polyclonal murine antibodies to TNF as being useful for diagnostic immunoassays of bacterial infections and certain pathologies, such as Kawasaki's pathology. Liang, et al., Biochem. Biophys. Res. Comm., 137:847-854 (1986), described rodent or murine moloclonal antibodies specific for recombinant human TNF, which has neutralizing activity in vitro. Fendly, et al., Hybridoma, 6:359-369 (1987), used such monoclonal antibodies to map epitopes of human TNF and to develop enzyme immunoassays.

A TNFα binding protein preparation has been commercialized by Immunex/Wyeth-Ayerst Laboratories, and is available under the trade name ENBREL® (etanercept) for the treatment of rheumatoid arthritis. Etanercept is a dimeric fusion protein consisting of the extracellular ligand binding portion of the human 75 kilodalton TNF receptor linked to the Fc portion of human IgG. The Fc component of enantercept contains the CH2 domain, the CH3 domain and hinge region, but not the CH1 domain of IgG1. The peptide is produced by recombinant DNA technology in a Chinese hamster ovary (CHO) mammalian cell expression system. It consists of 934 amino acids and has an apparent molecular weight of approximately 150 kilodaltons.

As shown by the work described above, significant progress has been made in the development and use of Cox-2 selective inhibitors and compounds that affect TNFα synthesis and activity. Moreover, the therapeutic potential of a combination of these two types of materials has been the subject of speculation (See. e.g., Scrivens, J., at Sharecast, http://www.sharecast.com/news/scnews1.asp?StoryID=8334, Jun. 7, 2001.

Accordingly, it would be useful to provide methods of therapy that are in addition to, and preferably an improvement upon, presently known methods for the prevention and treatment of pain, inflammation and inflammation related disorders, as well as the prevention and treatment of cardiovascular diseases and disorders and the prevention and treatment of cancer.

### SUMMARY OF THE INVENTION

Briefly, therefore the present invention is directed to a novel method for the treatment, prevention, or inhibition of pain, inflammation, or inflammation related disorder in a subject in need of such treatment, prevention, or inhibition, the method comprising administering to the subject a cyclooxygenase-2 selective inhibitor or prodrug thereof and a TNFα antagonist.

The present invention is also directed to a novel composition for the treatment, prevention, or inhibition or pain, inflammation, or inflammation-associated disorder comprising a combination which includes an amount of a TNFα antagonist and an amount of a cyclooxygenase-2 selective inhibitor or prodrug thereof which together constitute a pain or inflammation suppressing treatment or prevention effective amount of the combination.

The present invention is also directed to a novel pharmaceutical composition comprising a pharmaceutically-acceptable excipient and a combination which includes an amount of a TNFα antagonist and an amount of a cyclooxygenase-2 selective inhibitor or prodrug thereof which together constitute a pain or inflammation suppressing treatment or prevention effective amount of the combination.

The present invention is also directed to a novel kit that is suitable for use in the treatment, prevention or inhibition of pain, inflammation or inflammation-associated disorder, the kit comprises a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

The present invention is also directed to a novel method for the treatment, prevention, or inhibition of a cardiovascular disease or disorder in a subject in need of such treatment, prevention, or inhibition, the method comprising administering to the subject a cyclooxygenase-2 selective inhibitor or prodrug thereof and a TNFα antagonist

The present invention is also directed to a novel composition for the treatment, prevention, or inhibition of cardiovascular disease or disorder comprising a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

The present invention is also directed to a novel pharmaceutical composition for the treatment, prevention, or inhibition of cardiovascular disease or disorder comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

The present invention is also directed to a novel kit that is suitable for use in the treatment, prevention or inhibition of a cardiovascular disease or disorder, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of a cardiovascular disease or disorder.

The present invention is also directed to a novel method for the treatment, prevention, or inhibition of cancer in a subject in need of such treatment, prevention, or inhibition, the method comprising administering to the subject a cyclooxygenase-2 selective inhibitor or prodrug thereof and a TNFα antagonist which is selected from the group consisting of a compound that affects the synthesis of TNFα, a compound that inhibits the binding of TNFα with a receptor specific for TNFα, and a compound that interferes with intracellular signaling triggered by TNFα binding with a receptor.

The present invention is also directed to a novel composition for the treatment, prevention, or inhibition of cancer comprising a combination which includes an amount of a TNFα antagonist and an amount of a cyclooxygenase-2 selective inhibitor or prodrug thereof which together constitute an amount of the combination that is effective for the treatment, prevention, or inhibition of cancer.

The present invention is also directed to a novel pharmaceutical composition for the treatment, prevention, or inhibition of cancer comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of cancer.

The present invention is also directed to a novel kit that is suitable for use in the treatment, prevention or inhibition of cancer, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of cancer.

The present invention is also directed to a novel method for the treatment, prevention or inhibition of pain, inflammation or inflammation-related disorder in a subject in need of such treatment, prevention or inhibition, the method comprising administering to the subject a combination comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

a TNFα antagonist which comprises an antibody molecule having specificity for human TNFα, comprising the light chain variable region having the sequence given in SEQ ID NO:4 and the heavy chain variable region having the sequence given in SEQ ID NO:2.

The present invention is also directed to a novel method for the treatment, prevention or inhibition of pain, inflammation or inflammation-related disorder in a subject in need of such treatment, prevention or inhibition, the method comprising administering to the subject a combination comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

an antibody molecule having specificity for human TNFα, where the antibody molecule is a modified Fab fragment comprising a heavy chain having the sequence given in SEQ ID NO:6 and a light chain having the sequence given in SEQ ID NO:4.

The present invention is also directed to a novel method for the treatment, prevention or inhibition of pain, inflammation or inflammation-related disorder in a subject in need of such treatment, prevention or inhibition, the method comprising administering to the subject a combination comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a lysys-maleimide group wherein each amino group of the lysyl residue has covalently linked to it a poly(ethyleneglycol) or methoxypoly(ethyleneglycol) polymer.

The present invention is also directed to a novel therapeutic composition comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

a TNFα antagonist which comprises an antibody molecule having specificity for human TNFα, comprising the light chain variable region having the sequence given in SEQ ID NO:4 and the heavy chain variable region having the sequence given in SEQ ID NO:2.

The present invention is also directed to a novel therapeutic composition comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

an antibody molecule having specificity for human TNFα, where the antibody molecule is a modified Fab fragment comprising a heavy chain having the sequence given in SEQ ID NO:6 and a light chain having the sequence given in SEQ ID NO:4.

The present invention is also directed to a novel therapeutic composition comprising:

a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, BMS347070, meloxicam, prodrugs of any of them, and mixtures thereof; and

a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a poly(ethyleneglycol) or methoxypoly(ethyleneglycol) polymer.

Among the several advantages found to be achieved by the present invention, therefore, may be noted the provision of methods of therapy that are in addition to, and preferably an improvement upon, presently known methods for the prevention and treatment of pain, inflammation and inflammation related disorders, as well as the prevention and treatment of cardiovascular diseases and disorders and the prevention and treatment of cancer.

### DETAILED DESCRIPTION OF THE PREFERRED EMBODIMENTS

In accordance with the present invention, it has been discovered that pain, inflammation and inflammation-associated disorders, as well as cardiovascular diseases and disorders and cancer can be effectively prevented and/or treated in subjects that are in need of such prevention or treatment by treating the subject with a combination that includes a TNFα antagonist and one or more cyclooxygenase-2 selective inhibitors. The amount of the TNFα antagonist and the amount of the cyclooxygenase-2-selective inhibitor that are used in the treatment can be selected so that together they constitute a pain or inflammation suppressing treatment or prevention effective amount, or a cardiovascular disease or disorder treatment or prevention effective amount, or a cancer treatment or prevention effective amount.

The novel method of treating a subject with a combination of a TNFα antagonist and a cyclooxygenase-2-selective inhibitor provides a safe and efficacious method for preventing and alleviating pain and inflammation and for preventing and treating disorders that are associated with inflammation, as well as for treating and prevention cardiovascular diseases and disorders and cancer. In addition to being an efficacious method and composition for preventing and/or alleviating such disorders in a treated subject, such method and composition can also provide desirable properties such as stability, ease of handling, ease of compounding, lack of side effects, ease of preparation or administration, and the like.

The novel method and compositions comprise the use of a TNFα antagonist and a cyclooxygenase-2 selective inhibitor in combination.

The TNFα antagonist of the present invention is a compound which is capable of, directly or indirectly, counteracting, reducing or inhibiting the biological activity of TNFα, or the activation of receptors therefor. Without limitation, the TNFα antagonist of the present invention can be a compound that affects the synthesis of TNFα, or one that affects the maturation of TNFα, or one that inhibits the binding of TNFα with a receptor specific for TNFα, or one that interferes with intracellular signaling triggered by TNFα binding with a receptor.

The terms “biological activity” refer to a biological function (either inhibitory or stimulatory) caused by a native or naturally-occurring molecule, such as Cox-2 or TNFα, other than the ability to serve as an antigen in the production of an antibody against an antigenic epitope possessed by a native or naturally-occurring polypeptide of the invention. Similarly, “immunological” activity refers to the ability to serve as an antigen in the production of an antibody against an antigenic epitope possessed by the antigen.

A TNFα antagonist may act by inhibiting the synthesis of TNFα at (1) transcription, (2) translation, or (3) as a result of a decrease of the mRNA half-life. One example of a compound that inhibits TNFα synthesis is rolipram, which is a selective inhibitor of phosphodiesterase 4 (PDE4), and which has been identified as a potential treatment of HIV. (U.S. Pat. No. 5,547,979). Analogs of rolipram having similar biological activity also have been reported. For example, a series of PDE4 inhibitors in which the pyrrolidone ring of rolipram is replaced with a substituted oxazolidone ring is described in U.S. Pat. No. 5,783,591. Other rolipram analogs that are described as having improved bioavailability and lessened side effects are described in Exp. Opin. Ther. Patents, 8(5):526 (1998). A series of 3,4-disubstituted phenylsulfonamide derivatives that function to inhibit PDE4 are described in U.S. Pat. No. 5,728,712.

Thalidomide is another rolipram analog that has been reported to selectively decrease the half-life of TNF mRNA (Moreira, A. L. et al, J. Exp. Med., 177:1675-1680 (1993)), and has been shown to have clinical benefits in rheumatoid arthritis (RA). Exp. Opin. Ther. Patents, 8(5):550 (1998). A series of PDE4 inhibitors based on structural activity relationships (SAR) with thalidomide, and described as inhibiting TNF and being useful for RA treatment was described in U.S. Pat. No. 5,635,517.

Other PDE inhibitors that are useful TNFα antagonists include xanthine type compounds such as pentoxyfylline and theophylline. Analogs of pentoxyfylline that have been described for use in inflammatory diseases and central nervous system diseases are disclosed in U.S. Pat. No. 5,821,366.

The inhibition of PDE4 by using small molecule inhibitors is only one of many mechanisms known to inhibit the synthesis of TNF. For example, compounds that inhibit the production of protein kinase p38, which is important in TNF expression, have been described in U.S. Pat. Nos. 5,929,076; 5,916,891; 5,756,499; 5,739,143; 5,716,955; 5,670,527; 5,658,903; 5,593,992 and 5,593,991.

In addition to small molecule inhibitors, other agents are known to inhibit TNF synthesis. Examples of these inhibitors include cytokines such as interleukin 4, interleukin 10, transforming growth factor-beta and ciliary neutrotrophic factor, and other endogenous mediators such as prostanoids, corticosteroids, adenosine, histamine, nitric oxide, retinoic acid, and n-3 polyunsaturated fatty acids.

Other useful TNFα antagonists interfere with the maturation of TNF. Metalloprotease inhibitors that inhibit the activity of TNF converting enzyme (TACE) have been reported to interfere with TNF maturation. Exp. Opin. Ther. Patents, 8(5):537 (1998). Examples of these inhibitors are described in U.S. Pat. No. 5,872,146.

Suitable TNFα antagonists include compounds which inhibit the interaction of TNFα and a receptor therefor. The inhibition can be caused by physically blocking the receptor binding site on the cytokine itself, by blocking the ligand binding site on the receptor, or soluble TNF receptor can act as an antagonist by binding to TNF or competing with cell surface receptors for TNF binding. Particularly useful TNFα antagonists in the present invention include those which interfere with TNFα binding with the p55 receptor and the p75 receptor. TNFα antagonists that are specific for human TNFα are even more preferred. TNFα antagonists that inhibit TNFα-receptor binding can be a small molecule, peptide, protein, receptor extracellular domain, immunoadhesin, or an anti-TNFα antibody or fragment thereof.

The term “immunoadhesin” refers to a chimeric molecule which is a fusion of a ligand binding moiety, such as a receptor extracellular domain, with an immunoglobulin or a particular region of an immunoglobulin. For a bivalent form of an immunoadhesin, such a fusion could be to the Fc region of an IgG molecule. In a preferred embodiment, the immunoglobulin fusion includes the hinge, CH2 and CH3, or the hinge, CH1, CH2 and CH3 regions of an IgG1 molecule. U.S. Pat. No. 5,428,130 describes methods of production of immunoglobulin fusions. Immunoadhesins include antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody, and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA1, IgA2, IgE, IgD, or IgM.

Immunoadhesins that can serve as the subject TNFα antagonist include those that contain at least a TNFα binding portion of a TNFα receptor. Preferred immunoadhesins are described in U.S. Pat. Nos. 5,605,690; and 5,712,155. Other suitable TNFα antagonists are described in U.S. Pat. Nos. 5,482,130; 5,514,582; 5,336,603; and 5,565,335.

A “small molecule”, as those terms are used herein, means a molecule having a molecular weight below about 600 daltons, and is generally an organic molecule.

Antibodies acting specifically to block the receptor binding site on the TNF molecule include: 1) murine and human monoclonal antibodies, 2) chimeric antibodies, 3) humanized antibodies, and 4) antibody fragments. Certain other small molecules, such as oligonucleotides and peptides also can bind to the receptor binding domain of TNF. Chimeric antibodies generally comprise non-human variable regions and human constant regions. Humanized antibodies, on the other hand, are engineered by recombinant methodologies where only the antigen binding complementarity determining regions (CDR) are non-human in origin. Antibody fragments are the antigen binding domains with the minimum additional polypeptides to confer proper tertiary structure.

Antibodies that specifically inhibit the receptor binding site on TNF have been described in U.S. Pat. Nos. 5,656,272 and 5,919,452, which describe chimeric antibodies which inhibit the ligand binding site of the TNF receptor. The use of chimeric anti-TNF antibodies for the treatment of rheumatoid arthritis and Crohn's disease is described in U.S. Pat. Nos. 5,698,195 and 5,753,628. A neutralizing monoclonal antibody that is reactive with human TNF is described in U.S. Pat. No. 5,658,803. U.S. Pat. Nos. 5,994,510 and 5,654,407, and Fendly et al., Hybridoma, 6:359 (1987), and Bringman et al., Hybridoma, 6:489 (1987), describe other monoclonal antibodies specific to TNFα.

Although fewer references disclose TNFα antagonists that act by interfering with the TNF receptor, rather than with the TNF molecule, U.S. Pat. No. 5,359,037 describes inhibitor binding to the receptor site. Also, U.S. Pat. Nos. 5,981,701 and 5,695,953 describe non-proteolytic peptides capable of interacting with TNF to inhibit the binding of TNF to cells.

Soluble TNF receptors that competitively inhibit binding of TNF to its cell bound receptor have been the target of much recent work, For example, ENBREL® (etanercept), available from Immunex Corporation, Seattle, Wash., binds specifically with TNF and blocks it interaction with cell surface TNF receptors. This drug is currently available for human use for rheumatoid arthritis. The soluble, extracellular portions of both TNFR1 (p55) and TNFR2 (p75) naturally bind to TNFα and can be used, alone or bound to another molecule, as a TNFα antagonist of the present invention. See, e.g., Kohno et al., Proc. Natl. Acad. Sci. USA, 87:8331 (1990). TNFα antagonists that incorporate a soluble fragment of one or the other of these receptors are described in U.S. Pat. Nos. 5,482,130 and 5,514,582.

Once TNF binds to its receptor on the cell surface, a complex chain of signaling events occur. See, e.g., Exp. Opin. Ther. Patents, 8(10):1310 (1998). Several compounds have been described that inhibit TNF signaling. For example, polypeptides that inhibit binding to the intracellular domain of the p55 type (TNF-R1) TNF receptor and thus inhibit or modulate signal transduction by this receptor are described in U.S. Pat. Nos. 5,948,638; 5,891,675; 5,852,173; 5,849,501; 5,843,675; and 5,712,381. Other similar compounds are described in U.S. Pat. Nos. 5,563,039; 5,789,550; and 5,708,142.

Other suitable TNFα antagonists include compounds that reduce the levels of TNFα in tissues, and include the compounds described in U.S. Pat. Nos. 5,994,620; 5,981,701; 5,594,106; 5,336,603 and 4,565,397.

Table 1 shows U.S. Patents that describe TNF inhibitors that can be useful as the TNFα antagonist of the present invention. The category of the mode of action for each of the inhibitors, as has been discussed above, is indicated in the table.

TABLE 1
TNF inhibitors and mode of action category.

| Cate- | U.S. Pat. | |
|---|---|---|
| gory | No. | Title |
| 1 | 5,977,119 | Trisubstituted thoxanthines |
| 1 | 5,977,103 | Substituted imidazole compounds |
| 1 | 5,972,927 | Diazepinoindoles as phosphodiesterase 4 inhibitors |
| 1 | 5,962,478 | Inhibition of tumor necrosis factor alpha |
| 1 | 5,958,953 | Substituted indazole derivatives |
| 1 | 5,955,480 | Triaryl substituted imidazoles, compositions |
| | | containing such compounds and methods of use |
| 1 | 5,948,786 | Piperidinylpyrimidine derivatives |
| 1 | 5,939,422 | Chemical compounds having PDE-IV inhibition |
| | | activity |
| 1 | 5,939,421 | Quinazoline analogs and related compounds and |
| | | methods for treating inflammatory conditions |
| 1 | 5,935,978 | Compounds containing phenyl linked to aryl or |
| | | heteroaryl by an aliphatic- or heteroatom-containing |
| | | linking group |
| 1 | 5,935,966 | Pyrimidine carboxylates and related compounds and |
| | | methods for treating inflammatory conditions |
| 1 | 5,932,576 | 3(5)-heteroaryl substituted pyrazoles as p38 kinase |
| | | inhibitors |
| 1 | 5,932,425 | Compositions and methods for modulating cellular NF- |
| | | kappa-B activation |
| 1 | 5,929,076 | Cycloalkyl substituted imidazoles |
| 1 | 5,922,751 | Aryl pyrazole compound for inhibiting |
| | | phosphodiesterase IV and methods of using same |
| 1 | 5,919,801 | N-substituted peperidines as PDE4 inhibitors |
| 1 | 5,916,891 | Pyrimidinyl imidazoles |
| 1 | 5,905,089 | Use of sesquiterpene lactones for treatment of severe |
| | | inflammatory disorders |
| 1 | 5,891,924 | Curcumin (diferuloylmethane) inhibition of |
| | | NF-kappa-B activation |
| 1 | 5,891,904 | Use of inhibitors of phosphodiesterase IV |
| 1 | 5,889,014 | Heterocyclic compounds for inhibiting |
| | | phosphodieterase IV |
| 1 | 5,888,969 | Use of cytokine restraining agents to treat |
| | | inflammatory bowel disease |
| 1 | 5,869,516 | 4-(arylaminomethylene)-2,4-dihydro-3-pyrazolones |
| 1 | 5,869,511 | Isoxazoline compounds as inhibitors of TNF release |
| 1 | 5,864,037 | Methods for the synthesis of chemical compounds |
| | | having PDE-IV inhibitory activity |
| 1 | 5,864,028 | Degradation resistant mRNA derivatives linked to |
| | | TNF-alpha ribozymes |
| 1 | 5,861,382 | Methods for regulation of active TNF-alpha |
| 1 | 5,856,498 | Method of preparing phosphodiestrase IV inhibitors |
| 1 | 5,840,724 | Compounds containing phenyl linked to aryl or |
| | | heteroaryl by an aliphatic-o r |
| 1 | 5,834,485 | Quinoline sulfonamides and their therapeutic use |
| 1 | 5,811,300 | TNF-alpha ribozymes |
| 1 | 5,808,082 | Method of preparing phosphodiesterase IV inhibitors |
| 1 | 5,804,588 | Quinoline carboxamides and their therapeutic use |
| 1 | 5,792,774 | Quinolones and their therapeutic use |
| 1 | 5,783,591 | Administration of oxazolidinone and pyrolidinone |
| | | compounds for the treatment of inflammation |
| 1 | 5,776,954 | Substituted pyridyl pyrroles, compositions containing |
| | | such compounds and methods of use |
| 1 | 5,773,467 | Benzofuran sulphonamides |
| 1 | 5,756,499 | Substituted imidazole compounds |
| 1 | 5,739,143 | Imidazole compounds and compositions |
| 1 | 5,731,343 | Method of use of radicicol for treatment of |
| | | immunopathological disorders |
| 1 | 5,728,838 | Method of preparing phosphodiesterase IV inhibitors |
| 1 | 5,719,283 | Intermediates useful in the synthesis of |
| | | pyrazolopyrimidinone antianginal agents |
| 1 | 5,717,100 | Substituted imidazoles having anti-cancer and cytokine |
| | | inhibitory activity |
| 1 | 5,716,967 | Isoxazoline compounds as anti-inflammatory agents |
| 1 | 5,716,955 | Substituted imidazole compounds |
| 1 | 5,710,180 | Phenethylamine compounds |
| 1 | 5,710,170 | Tri-aryl ethane derivatives as PDE IV inhibitors |
| 1 | 5,710,160 | Diphenyl pyridyl ethane derivatives as PDE IV |
| | | inhibitors |
| 1 | 5,686,434 | 3-aryl-2-isoxazolines as inflammatory agents |
| 1 | 5,679,684 | Hydroxyalkylammonium-pyrimidines and nucleoside |
| | | derivatives, useful as inhibitors of inflammatory |
| | | cytokines |
| 1 | 5,670,527 | Pyridyl imidazole compounds and compositions |
| 1 | 5,658,949 | Inhibition of tumor necrosis factor by retinoic acid |
| 1 | 5,658,903 | Imidazole compounds, compositions and use |
| 1 | 5,648,359 | Tumor necrosis factor production inhibitors |
| 1 | 5,646,154 | Pharmaceutical compositions for inhibiting the |
| | | formation of tumor necrosis factor |
| 1 | 5,643,893 | N-substituted-(Dihydroxyboryl)alkyl purine, indole |
| | | and pyrimidine derivatives, useful as inhibitors of |
| | | inflammatory cytokines |
| 1 | 5,631,286 | Compounds useful for treating allergic or |
| | | inflammatory diseases |
| 1 | 5,624,913 | Method reducing TNF-alpha in mammals with cerebral |
| | | malaria |
| 1 | 5,622,977 | Tri-substituted (aryl or heteroaryl) derivatives and |
| | | pharmaceutical compositions containing the same |
| 1 | 5,616,490 | Ribozymes targeted to TNF-alpha RNA |
| 1 | 5,593,992 | Compounds (Novel 1,4,5-substituted imidazole |
| | | compounds and compositions for use in therapy as |
| | | cytokine inhibitors) |
| 1 | 5,593,991 | Imidazole compounds, use and process of making |
| 1 | 5,550,132 | Hydroxyalkylammonium-pyrimidines or purines and |
| | | nucleoside derivatives, useful as inhibitors of |
| | | inflammatory cytokines |
| 1 | 5,547,979 | TNF inhibition |
| 1 | 5,541,219 | 1-Alkoxy-2-(alkoxy- or cycloalkoxy-)-4- |
| | | (cyclothioalkyl- or cyclothioalkenyl-) benzenes as |
| | | inhibitors of cyclic AMP phosphodiesterase and |
| | | tumor necrosis factor |
| 1 | 5,496,855 | Anti-inflammatory compounds |
| 1 | 5,470,882 | Anti-inflammatory compounds |
| 1 | 5,447,957 | Anti-inflammatory compounds |
| 1 | 5,393,788 | Phenylalkyl oxamides |
| 1 | 5,385,901 | Method of treating abnormal concentrations of TNF- |
| | | alpha |
| 1 | 5,317,019 | Inhibition of interleukin-1 and tumor necrosis factor |
| | | production by monocytes and/or macrophages |
| 1 | 5,304,634 | Inhibitors for the formation of tumor necrosis factor |
| 1 | 5,096,906 | Method of inhibiting the activity of leukocyte derived |
| | | cytokines |
| 2 | 5,977,408 | Preparation and use of beta-sulfonamido hydroxamic |
| | | acids as matrix metalloproteinase and TACE inhibitors |
| 2 | 5,962,529 | Metalloproteinase inhibitors |
| 2 | 5,962,481 | Preparation and use of ortho-sulfonamido heteroaryl |
| | | hydroxamic acids as matrix metalloproteinase and tace |
| | | inhibitors |
| 2 | 5,955,435 | Peptidyl compounds having MMP and TNF inhibitory |
| | | activity |
| 2 | 5,952,320 | Macrocyclic inhibitors of matrix metalloproteinases |
| | | and TNF-alpha secretion |
| 2 | 5,932,695 | Synthesis of hydroxamic acid derivatives |
| 2 | 5,932,595 | Matrix metalloprotease inhibitors |
| 2 | 5,929,278 | Inhibitors of metalloproteases, pharmaceutical |
| | | compositions comprising same and methods of their |
| | | use |
| 2 | 5,929,097 | Preparation and use of orth-sulfonamido aryl |
| | | hydroxamic acids as matrix metalloproteinase and |
| | | tace inhibitors |
| 2 | 5,919,940 | Metalloproteinase inhibitors |
| 2 | 5,917,090 | Matrix metalloproteinase inhibitors |
| 2 | 5,908,851 | Derivatives of succinamide and their use as |
| | | metalloproteinase inhibitor |
| 2 | 5,902,791 | Metalloproteinase inhibitors |
| 2 | 5,891,878 | Quinolones and their therapeutic use |
| 2 | 5,883,241 | DNA sequence coding for a human metalloproteinase |
| | | and variants thereof |
| 2 | 5,872,152 | Use of MMP inhibitors |
| 2 | 5,872,146 | Mercapto alkyl peptidyl compounds having MMP and |
| | | TNF inhibitory activity |
| 2 | 5,866,717 | Metalloproteinase inhibitors |
| 2 | 5,866,588 | Imidazopyridine derivatives |
| 2 | 5,863,949 | Arylsulfonylamino hydroxamic acid derivatives |
| 2 | 5,861,436 | Hydroxamic acid derivatives as metalloproteinase |
| | | inhibitors |
| 2 | 5,859,253 | Metalloproteinase inhibitors |
| 2 | 5,853,977 | Mammalian TNF-alpha convertases |
| 2 | 5,853,623 | Peptidyl compounds and their therapeutic use as |
| | | inhibitors of metalloproteinases |
| 2 | 5,849,951 | Synthesis of carboxylic and hydroxamic acid |
| | | derivatives |
| 2 | 5,840,939 | Derivatives of succinamide and their use as |
| | | metalloproteinase inhibitors |
| 2 | 5,830,742 | TNF-alpha converting enzyme |
| 2 | 5,821,366 | Xanthines and their therapeutic use |
| 2 | 5,821,262 | Hydrozamic acid derivatives as inhibitors of cytokine |
| | | production |
| 2 | 5,817,822 | Certain alpha-azacycloalkyl substituted |
| | | arylsulfonamido acetohydroxamic acids |
| 2 | 5,792,774 | Quinolones and their therapeutic use |
| 2 | 5,770,624 | Certain alpha-substituted arylsulfonamido |
| | | acetohydroxamic acids |
| 2 | 5,763,621 | Metalloproteinase inhibitors |
| 2 | 5,753,666 | Quinolones and their therapeutic use |
| 2 | 5,753,653 | Metalloproteinase inhibitors, pharmaceutical |
| | | compositions containing them and their pharmaceutical |
| | | uses |
| 2 | 5,747,514 | Metalloproteinase inhibitors |
| 2 | 5,728,712 | 3,4-disubstituted-phenylsulphonamides and their |
| | | therapeutic use |
| 2 | 5,703,092 | Hydroxamic acid compounds as metalloproteinase and |
| | | TNF inhibitors |
| 2 | 5,700,838 | Hydroxamic acid derivatives as metalloproteinase |
| | | inhibitors |
| 2 | 5,698,706 | Heterocyclic amides and methods of use |
| 2 | 5,696,082 | Hydroxamic acid derivatives |
| 2 | 5,691,382 | Inhibition of TNF production with matrix |
| | | metaloproteinase inhibitors |
| 2 | 5,691,381 | Hydroxamic and carbocyclic acids as metalloprotease |
| | | inhibitors |
| 2 | 5,652,262 | Hydroxamic acid derivatives as metalloproteinase |
| | | inhibitors |
| 2 | 5,629,285 | Inhibitors of TNF-alpha secretion |
| 2 | 5,594,106 | Inhibitors of TNF-alpha secretion |
| 2 | 5,422,425 | Methods for the identification of cytokine convertase |
| | | inhibitors |
| 3 | 5,981,701 | Tumor necrosis factor inhibitory protein and its |
| | | purification |
| 3 | 5,972,599 | High affinity nucleic acid ligands of cytokines |
| 3 | 5,959,087 | Tumor necrosis factor binding ligands |
| 3 | 5,958,409 | Method for treating multiple sclerosis |
| 3 | 5,919,452 | Methods of treating TNF-alpha-mediated disease using |
| | | chimeric anti-TNF antibodies |
| 3 | 5,859,205 | Humanised antibodies |
| 3 | 5,795,967 | Tumor necrosis factor antagonists and their use |
| 3 | 5,753,628 | Peptide inhibitors of TNF containing predominantly D- |
| | | amino acids |
| 3 | 5,750,105 | Recombinant antibodies for human therapy |
| 3 | 5,702,705 | Antibody methods for the treatment of a hormone- |
| | | mediated disease |
| 3 | 5,700,466 | Method of ameliorating or preventing septic shock |
| | | using a monoclonal antibody specific to |
| | | cachectin/tumor necrosis factor |
| 3 | 5,698,419 | Monoclonal antibodies to cachetin/tumor necrosis |
| | | factor and methods for preparing same |
| 3 | 5,698,195 | Methods of treating rheumatoid arthritis using chimeric |
| | | anti-TNF antibodies |
| 3 | 5,695,953 | DNA that encodes a tumor necrosis factor inhibitory |
| | | protein and a recombinant method of production |
| 3 | 5,672,347 | Tumor necrosis factor antagonists and their use |
| 3 | 5,658,803 | Monoclonal antibodies reactive with cachectin |
| 3 | 5,658,570 | Recombinant antibodies for human therapy |
| 3 | 5,656,272 | Methods of treating TNF-alpha-mediated Crohn's |
| | | disease using chimeric anti-TNF antibodies |
| 3 | 5,654,407 | Human anti-TNF antibodies |
| 3 | 5,644,034 | Tumor necrosis factor binding ligands |
| 3 | 5,641,751 | Tumor necrosis factor inhibitors |
| 3 | 5,616,321 | Method of treating bacterial meningitis with anti-tumor |
| | | necrosis factor antibody |
| 3 | 5,519,000 | Tumor necrosis factor inhibitors |
| 3 | 5,506,340 | Tumor necrosis factor inhibitors |
| 3 | 5,486,595 | Tumor necrosis factor inhibitors |
| 3 | 5,436,154 | Monoclonal antibodies against human Tumor Necrosis |
| | | Factor alpha |
| 3 | 5,360,716 | Human tumor necrosis factor alpha specific |
| | | monoclonal antibody and method for detecting human |
| | | tumor necrosis factor alpha |
| 3 | 5,344,915 | Proteins and the preparation thereof |
| 3 | 5,231,024 | Monoclonal antibodies against human tumor necrosis |
| | | factor (TNF) and use thereof |
| 3 | 5,183,657 | Antibodies for use in antilymphocyte antibody therapy |
| 3 | 5,118,500 | Pharmaceutical containing TNF inhibitor |
| 3 | 5,075,236 | Method of detecting Kawasaki disease using |
| | | anti-tumor necrosis antibody |
| 3 | 5,766,917 | Method for identifying and producing a protease |
| | | capable of cleaving the TNF receptor |
| 3 | 5,665,859 | Molecules influencing the shedding of the TNF |
| | | receptor, their preparation and their use |
| 3 | 5,359,037 | Antibodies to TNF binding protein I |
| 3 | 5,945,397 | Purified p75 (type II) tumor necrosis factor receptor |
| | | polypeptides |
| 3 | 5,808,029 | DNA encoding a human TNF binding protein |
| 3 | 5,698,195 | Methods of treating rheumatoid arthritis using chimeric |
| | | anti-TNF antibodies |
| 3 | 5,610,279 | Human TNF receptor |
| 3 | 5,605,690 | Methods of lowering active TNF-alpha levels in |
| | | mammals using tumor necrosis factor receptor |
| 3 | 5,512,544 | Pharmaceutical compositions comprising an |
| | | anticytokine |
| 3 | 5,478,925 | Multimers of the soluble forms of TNF receptors, their |
| | | preparation and pharmaceutical compositions |
| | | containing them |
| 3 | 5,447,851 | DNA encoding a chimeric polypeptide comprising the |
| | | extracellular domain of TNF receptor fused to I_{g}G, |
| | | vectors, and host cells |
| 3 | 5,395,760 | DNA encoding tumor necrosis factor-alpha and -beta |
| | | receptors |
| 3 | 5,136,021 | TNF-inhibitory protein and a method of production |
| 4 | 5,948,638 | TNF receptor death domain ligand proteins |
| 4 | 5,891,719 | IKAP nucleic acids |
| 4 | 5,843,943 | Compounds for inhibition of ceramide-mediated signal |
| | | transduction |
| 4 | 5,834,435 | Inhibition of TNF-alpha pleiotropic and cytotoxic |
| | | effects |
| 4 | 5,789,550 | TRAF inhibitors |
| 4 | 5,712,381 | MADD, a TNF receptor death domain ligand protein |
| 4 | 5,708,142 | Tumor necrosis factor receptor-associated factors |
| 4 | 5,563,039 | TNF-receptor-associated intracellular signaling |
| | | proteins and methods of use |

Notes:
Key for category of TNFα antagonist:
1 Interference with TNF synthesis
2 Interference with maturation of TNF
3 Interference with receptor binding
4 Interference with receptor signaling

Preferred TNFα antagonists for the present invention include ENBREL® (etanercept) from Wyeth-Ayerst Laboratories/Immunex; REMICADE®, infliximab, which is an anti-TNF chimeric Mab (Centocor; Johnson & Johnson); anti-TNFα, D2E7 human Mab (Cambridge Antibody Technology); CDP-870, which is a PEGylated antibody fragment (Celltech) that is further described below; CDP-571, Humicade, which is a humanized Mab described in U.S. Pat. No. 5,994,510 (Celltech); PEGylated soluble TNFα Receptor-1 (Amgen); TBP-1, which is a TNF binding protein (Ares Serono); PASSTNF-alpha®, which is an anti-TNFα polyclonal antibody (Verigen); AGT-1, which is a mixture of three anti-cytokine antibodies to IFN-α, IFN-γ, and TNF (Advanced Biotherapy Concepts); TENEFUSE®, ienercept, which is a TNFR-Ig fusion protein (Roche); CytoTAb® (Protherics); TACE, which is a small molecule TNFα converting enzyme inhibitor (Immunex); small molecule TNF mRNA synthesis inhibitor (Nereus); PEGylated p75 TNFR Fc mutein (Immunex); AND TNFα antisense inhibitor.

Preferred TNFα antagonists that are useful in the present invention are disclosed in Great Britain Patent Application No. GB 0013810.7, filed Jun. 6, 2000, and in U.S. patent application Ser. No. 09/875,221, filed Jun. 6, 2001. Both of which applications are incorporated by reference herein in their entireties. Among the preferred TNFα antagonists that are disclosed is CDP-870, which has been mentioned above. CDP-870 is described as an antibody molecule having specificity for human TNFα.

Preferred antibody molecules that can act as the TNFα antagonist of the present invention may comprise: a complete antibody molecule, having full length heavy and light chains; a fragment thereof, such as a Fab, modified Fab, Fab′, F(ab′)₂ or Fv fragment; a light chain or heavy chain monomer or dimer; a single chain antibody, e.g., a single chain Fv in which the heavy and light chain variable domains are joined by a peptide linker. Similarly, the heavy and light chain variable regions may be combined with other antibody domains as appropriate.

Preferably, the antibody molecule that can act as the TNFα antagonist of the present invention is a Fab fragment. Preferably, the Fab fragment has a heavy chain having the sequence given as SEQ ID NO:2 and a light chain having the sequence given as SEQ ID NO:4. The amino acid sequences given in SEQ ID NO:2 and SEQ ID NO:4 are preferably encoded by the nucleotide sequences given in SEQ ID NO:1 and SEQ ID NO:3, respectively.

The residues in the sequences referred to herein are conventionally numbered according to a system devised by Kabat et al. (See, e.g., Kabat et al., in Sequences of Proteins of Immunological Interest, U.S. Department of Health and Human Services, NIH, Washington, D.C. (1987).

Alternatively, it is preferred that the antibody molecule that can be used as the TNFα antagonist of the present invention is a modified Fab fragment wherein the modification is the addition to the C-terminal end of its heavy chain of one or more amino acids to allow the attachment of an effector or reporter molecule. Preferably, the additional amino acids form a modified hinge region containing one or two cysteine residues to which the effector or reporter molecule may be attached. Such a modified Fab fragment preferably has a heavy chain having the sequence given as SEQ ID NO:6 and the light chain having the sequence given as SEQ ID NO:4. The amino acid sequence given in SEQ ID NO:6 is preferably encoded by the nucleotide sequence given in SEQ ID NO:5.

A preferred effector group is a polymer molecule, which may be attached to the modified Fab fragment to increase it half-life in-vivo. Particularly preferred polymer molecules include a polyalkylene polymer, such as a poly(ethyleneglycol) or, especially, a methoxypoly(ethyleneglycol) or a derivative thereof, and especially with a molecular weight in the range from and 25,000 Da to about 40,000 Da.

The preferred modified Fab fragment that can serve as the TNFα antagonist of the present invention is PEGylated (i.e., has poly(ethyleneglycol) covalently attached thereto) according to the method disclosed in EP-A-0948544.

The compound CDP-870 comprises a PEGylated Fab fragment wherein the heavy chain of the antibody part has the sequence given as SEQ ID NO:6 and the light chain has the sequence given as SEQ ID NO:4.

Another component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1, preferably greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity.

Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

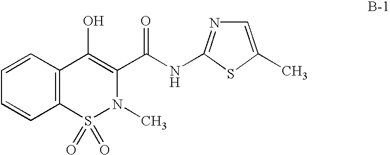

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

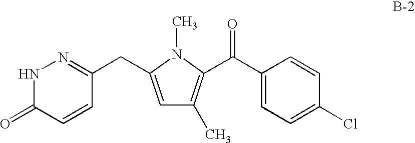

In a preferred embodiment of the invention the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, or III, shown below, and possessing, by way of example and not limitation, the structures disclosed in Table 2, including the diastereomers, enantiomers, racemates, tautomers, salts, esters, amides and prodrugs thereof.

Benzopyran Cox-2 selective inhibitors useful in the practice of the present invention are described in U.S. Pat. No. 6,034,256 and 6,077,850.

Formula I is:

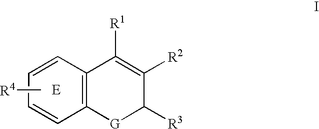

wherein G is selected from the group consisting of O or S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein R⁴ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with ring E forms a naphthyl radical; or an isomer or pharmaceutically acceptable salt thereof; and

including the diastereomers, enantiomers, racemates, tautomers, salts, esters, amides and prodrugs thereof.

Formula II is:

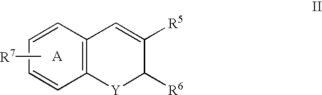

wherein:

Y is selected from the group consisting of O or S or NR^{b};

R^{b} is alkyl;

R⁵ is selected from the group consisting of carboxyl, aminocarbonyl, alkylsulfonylaminocarbonyl and alkoxycarbonyl;

R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;

or an isomer or pharmaceutically acceptable salt thereof.

The cyclooxygenase-2 selective inhibitor may also be a compound of Formula II, wherein:

Y is selected from the group consisting of oxygen and sulfur;

R⁵ is selected from the group consisting of carboxyl, lower alkyl, lower aralkyl and lower alkoxycarbonyl;

R⁶ is selected from the group consisting of lower haloalkyl, lower cycloalkyl and phenyl; and

R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R⁷ together with ring A forms a naphthyl radical; or an isomer or pharmaceutically acceptable salt thereof.

The cyclooxygenase-2 selective inhibitor may also be a compound of Formula II, wherein:

R⁵ is carboxyl;

R⁶ is lower haloalkyl; and

R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;

or an isomer or pharmaceutically acceptable salt thereof.

The cyclooxygenase-2 selective inhibitor may also be a compound of Formula II, wherein:

R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;

or an isomer or pharmaceutically acceptable salt thereof.

The cyclooxygenase-2 selective inhibitor may also be a compound of Formula II, wherein:

R⁶ is selected from the group consisting trifluoromethyl and pentafluoroethyl; and

R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical;

or an isomer or prodrug thereof.

The cyclooxygenase-2 selective inhibitor of the present invention can also be a compound having the structure of Formula III:

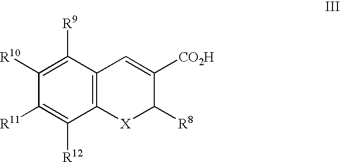

wherein:

X is selected from the group consisting of O and S;

R⁸ is lower haloalkyl;

R⁹ is selected from the group consisting of hydrido, and halo;

R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R¹¹ is selected from the group consisting of hydrido, lower alkyl, halo, lower alkoxy, and aryl; and

R¹² is selected from the group consisting of the group consisting of hydrido, halo, lower alkyl, lower alkoxy, and aryl; or an isomer or prodrug thereof.

The cyclooxygenase-2 selective inhibitor can also be a compound of having the structure of Formula III, wherein

R⁸ is selected from the group consisting of trifluoromethyl and pentafluoroethyl;

R⁹ is selected from the group consisting of hydrido, chloro, and fluoro;

R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R¹¹ is selected from the group consisting of hydrido, methyl, ethyl, isopropyl, tert-butyl, chloro, methoxy, diethylamino, and phenyl; and

R¹² is selected from the group consisting of hydrido, chloro, bromo, fluoro, methyl, ethyl, tert-butyl, methoxy, and phenyl; or an isomer or prodrug thereof.

TABLE 2
Examples of Chromene Cox-2 Selective Inhibitors as
Embodiments

| Compound | |
|---|---|
| Number | Structural Formula |
| B-3 | 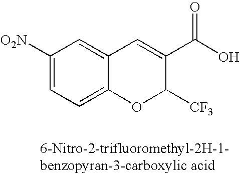 |
| B-4 | 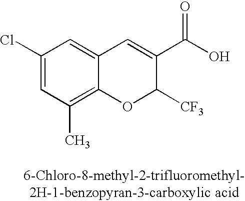 |
| B-5 | 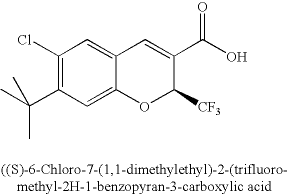 |
| B-6 | 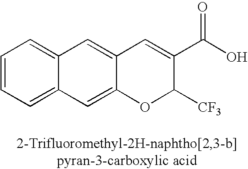 |
| B-7 | 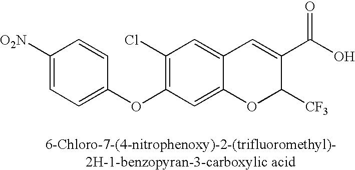 |
| B-8 | 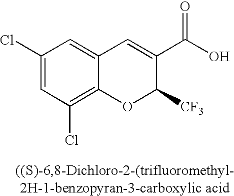 |
| B-9 | 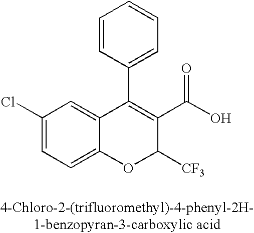 |
| B-10 | 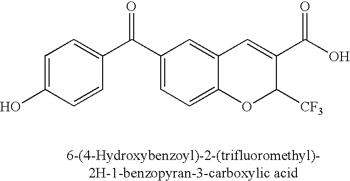 |
| B-11 | 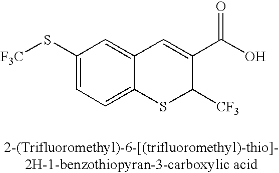 |
| B-12 | 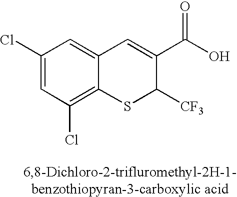 |
| B-13 | 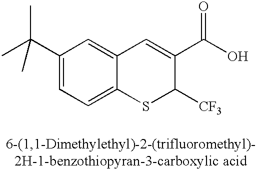 |
| B-14 | 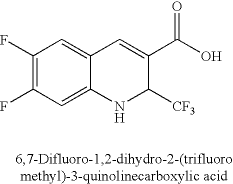 |
| B-15 | 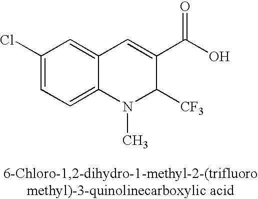 |
| B-16 | 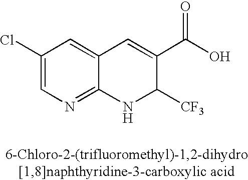 |
| B-17 | 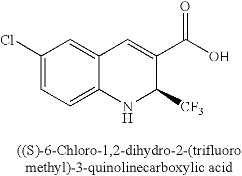 |

The compound SD-8381, shown as the structure in FIG. B-8, above, is a preferred chromene-type cyclooxygenase-2 selective inhibitor. The sodium salt form of the compound is preferred. Further information about SD-8381 can be found in U.S. Pat. No. 6,034,256.

Specific compounds that are useful for the cyclooxygenase-2 selective inhibitor include:

a1) 8-acetyl-3-(4-fluorophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-( 1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole,

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

i1) 1-[2-(4-methoxyphenyl)cyclopenten-1-yl]-4-(methylsulfonyl)benzene;

i2) 1-[2-(2,3-difluorophenyl)cyclopenten-1-yl]-4-(methylsulfonyl)benzene;

i3) 4-[2-(3-fluoro-4-methoxyphenyl)cyclopenten-1-yl]benzenesulfonamide;

i4) 1-[2-(3-chloro-4-methoxyphenyl)cyclopenten-1-yl]-4-(methylsulfonyl)benzene;

i5) 4-[2-(3-chloro-4-fluorophenyl)cyclopenten-1-yl]benzenesulfonamide;

i6) 4-[2-(2-methylpyridin-5-yl)cyclopenten-1-yl]benzenesulfonamide;

i7) ethyl 2-[4-(4-fluorophenyl)-5-[4-(methylsulfonyl) phenyl]oxazol-2-yl]-2-benzyl-acetate;

i8) 2-[4-(4-fluorophenyl)-5-[4-(methylsulfonyl)phenyl]oxazol-2-yl]acetic acid;

i9) 2-(tert-butyl)-4-(4-fluorophenyl)-5-[4-(methylsulfonyl)phenyl]oxazole;

i10) 4-(4-fluorophenyl)-5-(4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2 H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid;

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2 H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of Formula IV:

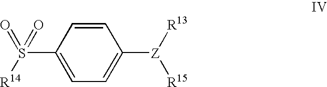

wherein:

Z is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula IV is selected from the group of compounds, illustrated in Table 3, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 3
Examples of Tricyclic COX-2 Selective
Inhibitors as Embodiments

| Compound | |
|---|---|
| Number | Structural Formula |
| B-18 | 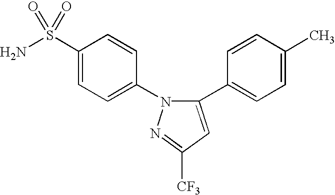 |
| B-19 | 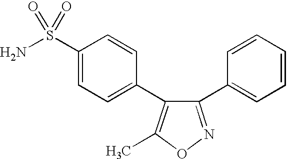 |
| B-20 | 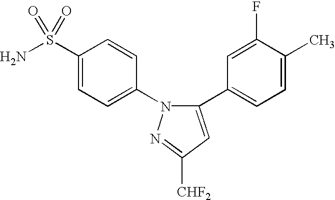 |
| B-21 | 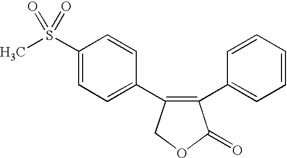 |
| B-22 | 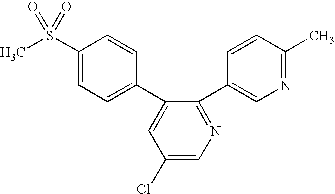 |
| B-23 | 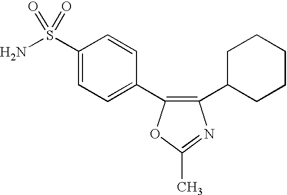 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-19, (See, e.g., U.S. Pat. No. 5,633,272), may be advantageously employed as a source of a cyclooxygenase inhibitor.

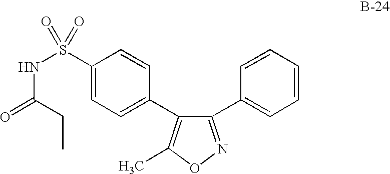

A preferred form of parecoxib is sodium parecoxib.

In another preferred embodiment of the invention, the compound ABT-963 having the formula B-25 that has been previously described in International Publication number WO 00/24719, is another tricyclic cyclooxygenase-2 selective inhibitor which may be advantageously employed.

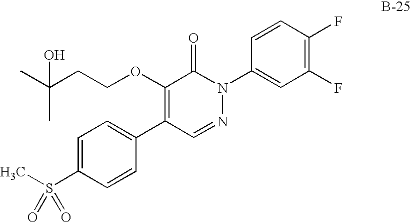

In a further preferred embodiment of the invention the cyclooxygenase inhibitor can be selected from the class of phenylacetic acid derivative cyclooxygenase-2 selective inhibitors represented by the general structure of Formula V:

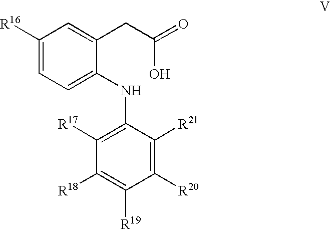

wherein R¹⁶ is methyl or ethyl;

R¹⁷ is chloro or fluoro;

R¹⁸ is hydrogen or fluoro

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor that is described in WO 99/11605 is a compound that has the designation of COX189 (CAS RN 346670-74-4), and that has the structure shown in Formula V,

wherein R¹⁶ is ethyl;

R¹⁷ and R¹⁹ are chloro;

R¹⁸ and R²⁰ are hydrogen; and

and R²¹ is methyl.

Compounds that have a structure similar to that shown in Formula V, which can serve as the Cox-2 selective inhibitor of the present invention, are described in U.S. Pat. Nos. 6,310,099 and 6,291,523.

Other preferred cyclooxygenase-2 selective inhibitors that can be used in the present invention have the general structure shown in formula VI, where the J group is a carbocycle or a heterocycle. Particularly preferred embodiments have the structure:

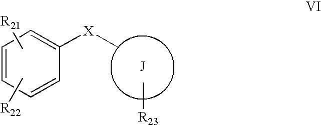

where:

X is O; J is 1-phenyl; R₂₁ is 2—NHSO₂CH₃; R₂₂ is 4—NO₂; and there is no R₂₃ group, (nimesulide), and

X is O; J is 1-oxo-inden-5-yl; R₂₁ is 2-F; R₂₂ is 4-F; and R₂₃ is 6—NHSO₂CH₃, (flosulide); and

X is O; J is cyclohexyl; R₂₁ is 2—NHSO₂CH₃; R₂₂ is 5—NO₂; and there is no R₂₃ group, (NS-398); and

X is S; J is 1-oxo-inden-5-yl; R₂₁ is 2-F; R₂₂ is 4-F; and R₂₃ is 6—N⁻SO₂CH₃·Na⁺,

(L-745337); and

X is S; J is thiophen-2-yl; R₂₁ is 4-F; there is no R₂₂ group; and R₂₃ is 5—NHSO₂CH₃, (RWJ-63556); and

X is O; J is 2-oxo-5(R)-methyl-5-(2,2,2-trifluoroethyl)furan-(5H)-3-yl; R₂₁ is 3-F; R₂₂ is 4-F; and R₂₃ is 4-(p—SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

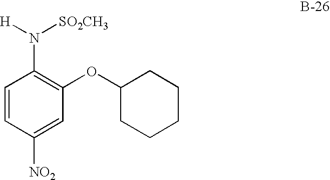

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

Other materials that can serve as he cyclooxygenase-2 selective inhibitor of the present invention include diarylmethylidenefuran derivatives that are described in U.S. Pat. No. 6,180,651. Such diarylmethylidenefuran derivatives have the general formula shown below in formula VII:

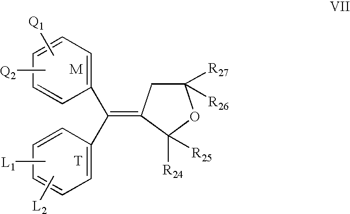

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q₁, Q₂, L₁ or L₂ is:

an —S(O)ₙ—R group, in which n is an integer equal to 0, 1 or 2 and R is a

lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q₁ and Q₂ or L₁ and L₂ are a methylenedioxy group; and

R₂₄, R₂₅, R₂₆ and R₂₇ independently are:

a hydrogen atom,

a halogen atom,

a lower alkyl radical having 1 to 6 carbon atoms,

a lower haloalkyl radical having 1 to 6 carbon atoms, or an aromatic radical selected from the group consisting of phenyl, naphthyl, thienyl, furyl and pyridyl; or,

R₂₄, R₂₅ or R₂₆, R₂₇ are an oxygen atom, or

R₂₄, R₂₅ or R₂₆, R₂₇, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Cox-2 selective inhibitors that are useful in the subject method and compositions can include the compounds that are described in U.S. Pat. Nos. 6,310,079; 6,306,890 and 6,303,628 (bicycliccarbonyl indoles); U.S. Pat. Nos. 6,133,292; 6,020,343; 5,981,576 ((methylsulfonyl)phenyl furanones); U.S. Pat. No. 6,083,969 (diarylcycloalkano and cycloalkeno pyrazoles); U.S. Pat. No. 6,077,869 (aryl phenylhydrazines); U.S. Pat. No. 6,071,936 (substituted pyridines); U.S. Pat. No. 6,307,047 (pyridazinone compounds); U.S. Pat. No. 6,140,515 (3-aryl-4-aryloxyfuran-5-ones); and U.S. Pat. Nos. 6,002,014; 5,994,381; and 5,945,539 (oxazole derivatives).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

One embodiment of the present invention includes a method for the treatment, prevention, or inhibition of pain, inflammation, or inflammation related disorder in a subject in need of such treatment, prevention, or inhibition. The method comprises administering to the subject a cyclooxygenase-2 selective inhibitor or prodrug thereof and a TNFα antagonist. It is preferred that the method comprises administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of pain, inflammation, or inflammation related disorder. The effective amount of the combination is preferably a therapeutically effective amount.

When the subject method is one that is directed to the treatment, prevention or inhibition of pain, inflammation, or inflammation-related disorder, the TNFα antagonist can be selected from the group consisting of a compound that affects the synthesis of TNFα, a compound that affects the maturation of TNFα, a compound that inhibits the binding of TNFα with a receptor specific for TNFα, and a compound that interferes with intracellular signaling triggered by TNFα binding with a receptor. TNFα antagonists that fall within these categories are described above.

In one embodiment, the present invention includes a composition for the treatment, prevention, or inhibition or pain, inflammation, or inflammation-associated disorder, where the composition comprises a combination which includes an amount of a TNFα antagonist and an amount of a cyclooxygenase-2 selective inhibitor or prodrug thereof which together constitute a pain or inflammation suppressing treatment or prevention effective amount of the combination.

In another embodiment, the present invention includes a pharmaceutical composition comprising a pharmaceutically-acceptable excipient and a combination which includes an amount of a TNFα antagonist and an amount of a cyclooxygenase-2 selective inhibitor or prodrug thereof which together constitute a pain or inflammation suppressing treatment or prevention effective amount of the combination.

In another embodiment, the present invention includes a kit that is suitable for use in the treatment, prevention or inhibition of pain, inflammation or inflammation-associated disorder, the kit comprises a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

The method and combination of the present invention are useful for, but not limited to, the prevention, inhibition, and treatment of pain and/or inflammation in a subject, and for treatment of inflammation-associated disorders, such as for use as an analgesic in the treatment of pain and headaches, or as an antipyretic for the treatment of fever. For example, combinations of the invention would be useful to treat arthritis, including, but not limited to, rheumatoid arthritis, spondyloarthopathies, gouty arthritis, osteoarthritis, systemic lupus erythematosus and juvenile arthritis. Such combinations of the invention would be useful in the treatment of asthma, bronchitis, menstrual cramps, tendinitis, bursitis, connective tissue injuries or disorders, and skin related conditions such as psoriasis, eczema, burns and dermatitis.

Combinations of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Combinations of the invention would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

Compositions having the novel combination would also be useful in the treatment of ophthalmic diseases, such as retinitis, retinopathies, conjunctivitis, uveitis, ocular photophobia, and of acute injury to the eye tissue. The compositions would also be useful in the treatment of pulmonary inflammation, such as that associated with viral infections and cystic fibrosis. The compositions would also be useful for the treatment of certain central nervous system disorders such as cortical dementias including Alzheimer's disease. The combinations of the invention are also useful as anti-inflammatory agents, such as for the treatment of arthritis.

As used herein, the terms “pain, inflammation or inflammation-associated disorder”, and “cyclooxygenase-2 mediated disorder” are meant to include, without limitation, each of the symptoms or diseases that is mentioned above.

The present method includes the treatment and/or prevention of a cyclooxygenase-2 mediated disorder in a subject, where the method comprises treating the subject having or susceptible to the disorder with a therapeutically-effective amount of a combination of a TNFα antagonist and a compound or salt of any of the cyclooxygenase-2 selective inhibitors that are described in this specification. This method is particularly useful where the cyclooxygenase-2 mediated disorder is inflammation, arthritis, pain, or fever.

In another embodiment, a method of the present invention includes a method for the treatment, prevention, or inhibition of a cardiovascular disease or disorder in a subject in need of such treatment, prevention, or inhibition, the method comprising administering to the subject a cyclooxygenase-2 selective inhibitor or prodrug thereof and a TNFα antagonist. It is preferred that the method comprises administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of a cardiovascular disease or disorder. Any TNFα antagonist that is described above can be used in this method, as can any of the cyclooxygenase-2 selective inhibitors that are described herein.

An embodiment of the present invention includes a composition for the treatment, prevention, or inhibition of cardiovascular disease or disorder comprising a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

Another embodiment of the present invention includes a pharmaceutical composition for the treatment, prevention, or inhibition of cardiovascular disease or disorder comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

In another embodiment, the present invention includes a kit that is suitable for use in the treatment, prevention or inhibition of a cardiovascular disease or disorder, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of a cardiovascular disease or disorder.

The compositions and methods described herein would be useful for, but not limited to, the prevention, treatment or amelioration of cardiovascular disease or disorder in a subject in need of such prevention, treatment or amelioration. Preferably, the compositions and methods described herein would be useful for the prevention, treatment or amelioration of inflammation-related cardiovascular disorders in a subject in need of such prevention, treatment or amelioration. The compositions and methods would be useful for prevention of coronary artery disease, aneurysm, arteriosclerosis, atherosclerosis including cardiac transplant atherosclerosis, myocardial infarction, embolism, stroke, thrombosis, including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

An embodiment of the present invention includes a method for the treatment, prevention, or inhibition of cancer in a subject in need of such treatment, prevention, or inhibition, the method comprising administering to the subject a cyclooxygenase-2 selective inhibitor or prodrug thereof and a TNFα antagonist which is selected from the group consisting of a compound that affects the synthesis of TNFα, a compound that inhibits the binding of TNFα with a receptor specific for TNFα, and a compound that interferes with intracellular signaling triggered by TNFα binding with a receptor. In this method, it is preferred that the method includes administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of cancer.

Compositions that are suitable for use in the method described just above include compositions for the treatment, prevention, or inhibition of cancer comprising a combination which include an amount of a TNFα antagonist and an amount of a cyclooxygenase-2 selective inhibitor or prodrug thereof which together constitute an amount of the combination that is effective for the treatment, prevention, or inhibition of cancer.

Also included as an embodiment of the present invention is a pharmaceutical composition for the treatment, prevention, or inhibition of cancer comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of cancer.

Another embodiment of the present invention includes a kit that is suitable for use in the treatment, prevention or inhibition of cancer, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of cancer.

The methods and compositions described herein as the subject methods and compositions would be useful for the prevention, treatment or amelioration of cancer. Preferably, the subject methods and compositions of the present invention may be used for the treatment, prevention or amelioration of neoplasia disorders. Such neoplasia disorders can include benign neoplasias, metastatic growths, and malignant neoplasias. Such neoplasias can include, for example, acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

In one preferred embodiment, the TNFα antagonist can include a compound that inhibits the interaction of TNFα and a receptor therefor. It is preferred that the TNFα antagonist is one that is capable of blocking the receptor binding site on TNFα, or by blocking the ligand binding site on the receptor. This can be accomplished by a TNFα antagonist which is capable of interfering with TNFα binding with the p55 receptor and the p75 receptor. Particularly useful are TNFα antagonists which are specific for human TNFα. An example of such a TNFα antagonist includes an antibody molecule, or fragment thereof, having specificity for human TNFα, comprising a light chain variable region having the amino acid sequence given in SEQ ID NO:4 and a heavy chain variable region having the amino acid sequence given in SEQ ID NO:2.

Another embodiment of the method includes a TNFα antagonist comprises a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a poly(ethyleneglycol) or methoxypoly(ethyleneglycol) residue having a molecular weight of about 20,000 Da.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are-intended to qualify the amount of each agent for use in the combination therapy which will achieve the goal of improvement in the severity of cancer, cardiovascular disease, or pain and inflammation and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp.1707-1711.

In the present methods, the amount of the TNFα antagonist that is used is such that, when administered with the cyclooxygenase-2 selective inhibitor, it is sufficient to constitute an effective amount of the combination. It is preferred that the dosage of the combination constitute a therapeutically effective amount.

It is preferred that the amount of the TNFα antagonist that is used in combination with a Cox-2 selective inhibitor for a single dosage of treatment is within a range of from about 0.1 mg/kg of body weight of the subject to about 100 mg/kg. It is more preferred that the amount is from about 1 mg/kg to about 80 mg/kg, even more preferred that it is from about 10 mg/kg to about 50 mg/kg, and yet more preferred that it is from about 15 mg/kg to about 30 mg/kg.

The frequency of dose will depend upon the half-life of the TNFα antagonist molecule. If the TNFα antagonist molecule has a short half life (e.g. from about 2 to 10 hours) it may be necessary to give one or more doses per day. Alternatively, if the TNFα antagonist molecule has a long half-life (e.g. from about 2 to about 15 days) it may only be necessary to give a dosage once per day, per week, or even once every 1 or 2 months.

The amount of Cox-2 selective inhibitor that is used in the subject method may be an amount that, when administered with the TNFα antagonist, is sufficient to constitute an effective amount of the combination. Preferably, such amount would be sufficient to provide a therapeutically effective amount of the combination. The therapeutically effective amount can also be described herein as a pain or inflammation suppressing treatment or prevention effective amount of the combination, or as a cardiovascular disorder or disease suppressing treatment or prevention effective amount, or as a cancer suppressing treatment or prevention effective amount.

In the present method, the amount of Cox-2 selective inhibitor that is used in the novel method of treatment preferably ranges from about 0.01 to about 100 milligrams per day per kilogram of body weight of the subject (mg/day.kg), more preferably from about 0.1 to about 50 mg/day.kg, even more preferably from about 1 to about 20 mg/day.kg.

When the Cox-2 selective inhibitor comprises rofecoxib, it is preferred that the amount used is within a range of from about 0.15 to about 1.0 mg/day.kg, and even more preferably from about 0.18 to about 0.4 mg/day.kg.

When the Cox-2 selective inhibitor comprises etoricoxib, it is preferred that the amount used is within a range of from about 0.5 to about 5 mg/day.kg, and even more preferably from about 0.8 to about 4 mg/day.kg.

When the Cox-2 selective inhibitor comprises parecoxib sodium, it is preferred that the amount used is within a range of from about 0.1 to about 3 mg/day.kg, and even more preferably from about 0.3 to about 1 mg/day.kg.

When the Cox-2 selective inhibitor comprises celecoxib, it is preferred that the amount used is within a range of from about 1 to about 10 mg/day.kg, even more preferably from about 1.4 to about 8.6 mg/day.kg, and yet more preferably from about 2 to about 3 mg/day.kg.

In the present method, and in the subject compositions, the TNFα antagonist is administered with, or is combined with, a Cox-2 selective inhibitor. It is preferred that the weight ratio of the amount of TNFα antagonist to the amount of Cox-2 selective inhibitor that is administered to the subject is within a range of from about 0.001:1 to about 100:1, more preferred is a range of from about 0.01:1 to about 10:1, even more preferred is a range of from about 0.1:1 to about 0.3:1.

In an embodiment of the present method, other compounds, such as chondroitin sulfate and glucosamine can be added as components of the combination with the cyclooxygenase-2 selective inhibitor and the TNFα antagonist. If glucosamine is used, the amount of glucosamine preferably ranges from about 0.1 to about 500 milligrams per day per kilogram of body weight of the subject (mg/day.kg), more preferably from about 0.5 to about 100 mg/day kg, even more preferably from about 1 to about 50 mg/day.kg, yet more preferably from about 5 to about 35 mg/day.kg, and even more preferably from about 15 to about 25 mg/day.kg. If chondroitin sulfate is used, the amount of chondroitin sulfate preferably ranges from about 5 to about 150 milligrams per day per kilogram of body weight of the subject (mg/day.kg), more preferably from about 8 to about 100 mg/day.kg, even more preferably from about 10 to about 30 mg/day.kg, and even more preferably from about 10 to about 20 mg/day.kg.

The combination of a TNFα antagonist and a Cox-2 selective inhibitor, can be supplied in the form of a novel therapeutic composition that is believed to be within the scope of the present invention. The relative amounts of each component in the therapeutic composition may be varied and may be as described just above. The TNFα antagonist and Cox-2 selective inhibitor, that are described above can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier, a pharmaceutical composition is formed. A pharmaceutical composition of the present invention is directed to a composition suitable for the prevention or treatment of pain, inflammation and/or an inflammation-associated disorder. The pharmaceutical composition comprises a pharmaceutically acceptable carrier, a TNFα antagonist, and a cyclooxygenase-2 selective inhibitor.

Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of TNFα antagonists and cyclooxygenase-2 selective inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of cancer, cardiovascular disease or disorder, or pain and/or inflammation associated with, but not limited to, any of the diseases or disorders described above. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has cancer, cardiovascular disease, or pain, inflammation and/or any one of the known inflammation-associated disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of cancer, cardiovascular disease, or pain, inflammation and/or an inflammation-associated disorder. The subject may be a human subject who is at risk for cancer, cardiovascular disease, or pain and/or inflammation, or for obtaining an inflammation-associated disorder, such as those described above. The subject may be at risk due to genetic predisposition, sedentary lifestyle, diet, exposure to disorder-causing agents, exposure to pathogenic agents and the like.

The pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of a cyclooxygenase-2 inhibitor agent and a TNFα antagonist, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

The phrase “therapeutically-effective” and “effective for the treatment, prevention, or inhibition”, are is intended to qualify the amount of each agent for use in the combination therapy which will achieve the goal of improvement in the severity of cancer, cardiovascular disease, or inflammation and the frequency of incidence over treatment of each agent by itself, while avoiding adverse side effects typically associated with alternative therapies.

Although the combination of the present invention may include administration of a TNFα antagonist component and a cyclooxygenase-2 selective inhibitor component within an effective time of each respective component, it is preferable to administer both respective components contemporaneously, and more preferable to administer both respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol an hydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. When the TNFα antagonist is an antibody or antibody fragment, reconstitution in sterile bacteriostatic water for injuction (USP (0.9% benzyl alcohol) can be used. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The present invention further comprises kits that are suitable for use in performing the methods of treatment, prevention or inhibition described above. In one embodiment, the kit contains a first dosage form comprising a TNFα antagonist in one or more of the forms identified above and a second dosage form comprising one or more of the cyclooxygenase-2 selective inhibitors or prodrugs thereof identified above, in quantities sufficient to carry out the methods of the present invention. Preferably, the first dosage form and the second dosage form together comprise a therapeutically effective amount of the compounds for the treatment, prevention, or inhibition of pain, inflammation or inflammation-associated disorder, or of cardiovascular disease or disorder, or of cancer.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### COMPARATIVE EXAMPLE 1

This example shows the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇ H₁₄ N₃ O₂ SF₃: C, 53.54; H, 3.70; N, 11.02. The composition that was found by analysis was: C, 53.17; H, 3.81; N, 10.90.

### EXAMPLE 2

This illustrates the production of an injectable therapeutic composition containing parecoxib sodium and etanercept and of a pharmaceutical composition containing the combination.

Etanercept is a TNF-specific antibody that binds specifically with TNFα and blocks its interaction with cell surface TNFα receptors. Etanercept is available under the trade name ENBREL® from Immunex Corporation, Seattle, Wash., or Wyeth-Ayerst Laboratories, Philadelphia, Pa. Parecoxib sodium can be prepared as described in U.S. Pat. No. 5,932,598, or it can be obtained from Pharmacia Corporation, Peapack, N.J.

A pharmaceutical composition of the present invention can be formed by intermixing etanercept (7 g), and parecoxib sodium (40 g) into a solution by mixing the two active ingredients with sterile bacteriostatic water for injection, USP, containing 0.9% benzyl alcohol, in an amount that is suitable for preparation of an injectable solution of the two active materials. For example, 1000 ml of the carrier solution can be used. The mixing and handling of the solution should take place under conditions that preserve the sterility of the solution. When the active ingredients have completely dissolved, the solution can be packaged into 1000 separate single use 1.0 ml injection vials. Each injection vial contains 1 ml of solution that contains 40 mg of parecoxib sodium and 7 mg of etanercept. A common dosage rate for this composition for an adult human would be about one injection per day.

### EXAMPLE 3

This illustrates the evaluation of the-biological efficacy of a therapeutic composition of etanercept and parecoxib sodium for the alleviation of pain and inflammation.

A therapeutic composition containing etanercept and parecoxib sodium is prepared as described in Example 2. The biological efficacy of the composition is determined by a rat carrageenan foot pad edema test and by a rat carrageenan-induced analgesia test.

Rat Carrageenan Foot Pad Edema Test:

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with the compounds of Example 2 suspended in a carrier vehicle containing 0.5% methylcellulose and 0.025% surfactant, or with only the carrier vehicle alone. One hour later, a subplantar injection of 0.1 mL of 1% solution of carrageenan/sterile 0.9% saline is administered to one foot and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDS, in Non-steroidal Anti-Inflammatory Drugs, (J. Lombardino, ed. 1985)). The percent inhibition shows the percent decrease from control paw volume determined in this procedure. It is believed that the data would show that the combination of etanercept and parecoxib sodium provides effective anti-inflammatory activity.

Rat Carrageenan-induced Analgesia Test:

The analgesia test using rat carrageenan is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special PLEXIGLAS® container with a transparent floor having a high intensity lamp as a radiant heat source, positionable under the floor. After an initial twenty-minute period, thermal stimulation is begun on either the injected foot or on the contralateral uninjected foot. A photoelectric cell will turn off the lamp and timer when the light is interrupted by paw withdrawal. The time until the rat withdraws its foot is then measured. The withdrawal latency in seconds is determined for the control and drug-treated groups, and percent inhibition of the hyperalgesic foot withdrawal is determined. It is believed that results would show that a combination of etanercept and parecoxib sodium provides effective analgesic activity.

### EXAMPLE 4

This illustrates the biological efficacy of a therapeutic composition of etanercept and parecoxib sodium for the treatment of collagen-induced arthritis in mice.

A therapeutic composition containing etanercept and parecoxib sodium is prepared as described in Example 2. The biological efficacy of the composition is determined by induction and assessment of collagen-induced arthritis in mice.

Arthritis is induced in 8-12 week old male DBA/1 mice by injection of 50 μg of chick-type II collagen (CII) in complete Freunds adjuvant (Sigma) on day 0 at the base of the tail as described in [J. Stuart, Annual Rev. Immunol., 2, 199 (1984)]. Compounds are prepared as a suspension in 0.5% methylcellulose (Sigma, St. Louis, Mo.), and 0.025% Tween 20 (Sigma). The cyclooxygenase-2 inhibitor (parecoxib sodium), and etanercept are administered alone or in combination as a therapeutic composition as described in Example 2. The compounds are administered in non-arthritic animals by gavage in a volume of 0.1 ml beginning on day 20 post collagen injection and continuing daily until final evaluation on day 55. Animals are boosted on day 21 with 50 μg of collagen (CII) in incomplete Freunds adjuvant. The animals are subsequently evaluated several times each week for incidence and severity of arthritis until day 56. Any animal with paw redness or swelling is counted as arthritic. Scoring of severity is carried out using a score of 0-3 for each paw (maximal score of 12/mouse) as described in P. Wooley, et al., Trans. Proc., 15, 180 (1983). The animals are measured for incidence of arthritis and severity in the animals where arthritis was observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw are scored as 1. Gross swelling of the whole paw or deformity is scored as 2. Ankylosis of joints is scored as 3.

Histological Examination of Paws:

In order to verify the gross determination of a non-arthritic animal, a histological examination can be performed. Paws from animals sacrificed at the end of the experiment are removed, fixed and decalcified as previously described [R. Jonsson, J. Immunol. Methods, 88, 109 (1986)]. Samples are paraffin embedded, sectioned, and stained with hematoxylin and eosin by standard methods. Stained sections are examined for cellular infiltrates, synovial hyperplasia, and bone and cartilage erosion.

It is believed that results will show that the combination of a cyclooxygenase-2 selective inhibitor with the TNFα antagonist etanercept was an efficacious treatment for collagen-induced arthritis in mice.

It is believed that Examples 2 through 4 can be repeated with compositions comprising any TNFα antagonist in combination with any of the cyclooxygenase-2 selective inhibitors that are described herein, and in particular with the combination of a cyclooxygenase-2 selective inhibitor and CDP-870, with the results showing that the combination provides effective anti-inflammatory activity, effective analgesic activity, and is an efficacious treatment of collagen-induced arthritis in mice.

### EXAMPLE 5

This example illustrates the efficacy of a TNFα antagonist in combination with a cyclooxygenase-2 selective inhibitor for the treatment of cancer.

A combination of any one or more of the TNFα antagonists that are described herein with any one or more of the cyclooxygenase-2 selective inhibitors that are described herein scan be prepared by the methods described in Example 2. The efficacy of the combination can be tested by the methods described in U.S. Pat. No. 6,242,196, for:

a. the reduction in size of adipose cell tumors in vivo;

b. the inhibition of proliferation of leukemic cells; and

c. the inhibition of proliferation of prostate cancer cells.

It is believed that the subject combinations would be found to be effective in reducing the size of adipose cell tumors in vivo; in inhibiting the proliferation of leukemic cells; and in inhibiting the proliferation of prostate cancer cells.

### EXAMPLE 6

This example illustrates the efficacy of a TNFα antagonist in combination with a cyclooxygenase-2 selective inhibitor for the improvement of cardiac function in myocardial infarction.

A combination of any one or more of the TNFα antagonists that are described herein with any one or more of the cyclooxygenase-2 selective inhibitors that are described herein can be prepared by the methods described in Example 2. The efficacy of the combination can be tested by the methods described by Saito, T. et al., in Biochem. and Biophys. Res. Communic., 273:772-775 (2000), for the improvement of cardiac function in myocardial infarction. It is believed that the subject combinations would be found to be effective in improving cardiac function in myocardial infarction.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method for the treatment, prevention, or inhibition of pain, inflammation, or inflammation related disorder in a subject in need of such treatment, prevention, or inhibition, the method comprising administering to the subject a cyclooxygenase-2 selective inhibitor or prodrug thereof and a TNFα antagonist.

2. The method according to claim 1, wherein the method comprises administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of pain, inflammation, or inflammation related disorder.

3. The method according to claim 2, wherein the effective amount of the combination is a therapeutically effective amount.

4. The method according to claim 1, wherein the TNFα antagonist is selected from the group consisting of a compound that affects the synthesis of TNFα, a compound that affects the maturation of TNFα, a compound that inhibits the binding of TNFα with a receptor specific for TNFα, and a compound that interferes with intracellular signaling triggered by TNFα binding with a receptor.

5. The method according to claim 4, wherein the TNFα antagonist comprises a compound-that inhibits the synthesis of TNFα.

6. The method according to claim 5, wherein the TNFα antagonist comprises a compound that inhibits the synthesis of TNFα where the compound is selected from the group consisting of rolipram, a selective inhibitor of phosphodiesterase 4, an analog of rolipram having similar biological activity, a 3,4-disubstituted phenylsulfonamide derivative that inhibits phosphodiesterase 4, thalidomide, a xanthine type compound that inhibits phosphodiesterase 4, pentoxyfylline, theophylline, an analog of pentoxyfylline that inhibits phosphodiesterase 4, a compound that inhibits the production of protein kinase p38, a cytokine which inhibits the synthesis of TNFα, interleukin 4, interleukin 10, transforming growth factor-beta, ciliary neutrotrophic factor, a prostanoid which inhibits the synthesis of TNFα, a corticosteroid which inhibits the synthesis of TNFα, adenosine, a histamine which inhibits the synthesis of TNFα, nitric oxide, retinoic acid, and n-3 polyunsaturated fatty acids which inhibit the synthesis of TNFα.

7. The method according to claim 4, wherein the TNFα antagonist comprises a compound that affects the maturation of TNFα.

8. The method according to claim 7, wherein the TNFα antagonist comprises a metalloproteinase inhibitor.

9. The method according to claim 8, wherein the TNFα antagonist comprises a compound that inhibits TNFα convertase.

10. The method according to claim 4, wherein the TNFα antagonist comprises a compound that inhibits the interaction of TNFα and a receptor therefor.

11. The method according to claim 10, wherein the TNFα antagonist is capable of blocking the receptor binding site on TNFα, or by blocking the ligand binding site on the receptor.

12. The method according to claim 11, wherein the TNFα antagonist is capable of interfering with TNFα binding with the p55 receptor and the p75 receptor.

13. The method according to claim 12, wherein the TNFα antagonist is specific for human TNFα.

14. The method according to claim 10, wherein the TNFα antagonist comprises a compound that is selected from, the group consisting of a small molecule, peptide, protein, receptor extracellular domain, immunoadhesin, and an anti-TNFα antibody or fragment thereof.

15. The method according to claim 14, wherein the TNFα antagonist comprises an antibody which is capable of acting specifically to block the receptor binding site on the TNFα molecule, and where the antibody is selected from the group consisting of murine and human monoclonal antibodies, chimeric antibodies, humanized antibodies, antibody fragments, oligonucleotides which are capable of binding to the receptor binding domain of TNFα, and peptides which are capable of binding to the receptor binding domain of TNFα.

16. The method according to claim 15, wherein the TNFα antagonist comprises etanercept.

17. The method according to claim 10, wherein the TNFα antagonist comprises an antibody molecule having specificity for human TNFα, comprising the light chain variable region having the sequence given in SEQ ID NO:4 and the heavy chain variable region having the sequence given in SEQ ID NO:2.

18. The method according to claim 10, wherein the TNFα antagonist comprises an antibody molecule having specificity for human TNFα, where the antibody molecule is a modified Fab fragment comprising a heavy chain having the sequence given in SEQ ID NO:6 and a light chain having the sequence given in SEQ ID NO:4.

19. The method according to claim 10, wherein the TNFα antagonist comprises a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a poly(ethyleneglycol) or methoxypoly(ethyleneglycol) polymer.

20. The method according to claim 4, wherein the TNFα antagonist comprises a compound that inhibits TNFα signaling.

21. The method according to claim 1, wherein the TNFα antagonist comprises a compound that is selected from the group consisting of etanercept, infliximab, anti-TNFα, D2E7 human Mab, CDP-870, humicade, PEGylated soluble TNFα Receptor-1, TBP-1, PASSTNF-alpha®, AGT-1, ienercept, CytoTAb®, TACE, small molecule TNF mRNA synthesis inhibitor, PEGylated p75 TNFR Fc mutein, and TNFα antisense inhibitor.

22. The method according to claim 1, wherein the cyclooxygenase-2 selective inhibitor or prodrug thereof has a cyclooxygenase-2 IC₅₀ of less than about 0.2 μmol/L.

23. The method according to claim 22, wherein the cyclooxygenase-2 selective inhibitor or prodrug thereof has a cyclooxygenase-1 IC₅₀ of at least about 1 μmol/L.

24. The method according to claim 1, wherein the cyclooxygenase-2 selective inhibitor is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, meloxicam, prodrugs of any of them, and mixtures thereof.

25. The method according to claim 24, wherein the cyclooxygenase-2 selective inhibitor comprises celecoxib or a prodrug thereof.

26. The method according to claim 1, wherein the cyclooxygenase-2 selective inhibitor comprises a material that is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, mixtures of any two or more thereof, and prodrugs thereof.

27. The method according to claim 26, wherein the cyclooxygenase-2 selective inhibitor comprises a material that is selected from the group consisting of NS-398, RWJ-63556, CS-502, LAS-34475, LAS-34555, S-33516, SD-8381, BMS-347070, S-2474, mixtures of any two or more thereof, and prodrugs thereof.

28. The method according to claim 1, wherein the amount of the TNFα antagonist is within a range of about 0.1 mg/kg of body weight of the subject per day (mg/kg.day) to about 100 mg/kg.day.

29. The method according to claim 28, wherein the amount of the TNFα antagonist is within a range of about 1 mg/kg.day to about 80 mg/kg.day.

30. The method according to claim 29, wherein the amount of the TNFα antagonist is within a range of about 10 mg/kg.day to about 50 mg/kg.day.

31. The method according to claim 30, wherein the amount of the TNFα antagonist is within a range of about 15 mg/kg.day to about 30 mg/kg.day.

32. The method according to claim 28, wherein the amount of the cyclooxygenase-2 selective inhibitor or prodrug thereof is within a range of from about 0.01 to about 100 mg/day per kg of body weight of the subject.

33. The method according to claim 32, wherein the amount of the cyclooxygenase-2 selective inhibitor or prodrug thereof is within a range of from about 1 to about 20 mg/day per kg of body weight of the subject.

34. The method according to claim 1, wherein the weight ratio of the amount of the TNFα antagonist to the amount of cyclooxygenase-2 selective inhibitor or prodrug thereof that is administered to the subject is within a range of from about 0.001:1 to about 100:1.

35. The method according to claim 34, wherein the weight ratio of the amount of the TNFα antagonist to the amount of cyclooxygenase-2 selective inhibitor or prodrug thereof that is administered to the subject is within a range of from about 0.01:1 to about 10:1.

36. The method according to claim 35, wherein the weight ratio of the amount of TNFα antagonist to the amount of cyclooxygenase-2 selective inhibitor or prodrug thereof that is administered to the subject is within a range of from about 0.1:1 to about 0.3:1.

37. The method according to claim 1, wherein the pain, inflammation or inflammation associated disorder is selected from the group consisting of headache, fever, arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, nervous system disorders, cortical dementias, and Alzheimer's disease.

38. The method according to claim 1, wherein the pain, inflammation or inflammation associated disorder is an opthalmic disease or opthalmic injury.

39. The method according to claim 38, wherein the opthalmic disease or opthalmic injury is selected from the group consisting of retinitis, retinopathies, conjunctivitis, uveitis, ocular photophobia, acute injury to the eye tissue,

40. The method according to claim 37, wherein the pain, inflammation or inflammation associated disorder is arthritis.

41. The method according to claim 40, wherein the arthritis is osteoarthritis.

42. The method according to claim 40, wherein the arthritis is rheumatoid arthritis.

43. The method according to claim 1, wherein the subject is an animal.

44. The method according to claim 43, wherein the subject is a human.

45. The method according to claim 1, wherein the treating step comprises administering a TNFα antagonist and a cycloxoygenase-2 selective inhibitor to the subject enterally or parenterally in one or more dose per day.

46. The method according to claim 45, wherein the TNFα antagonist and the cycoloxygenase-2 selective inhibitor are administered to the subject substantially simultaneously.

47. The method according to claim 45, wherein the TNFα antagonist and the cycoloxygenase-2 selective inhibitor are administered sequentially.

48. A composition for the treatment, prevention, or inhibition or pain, inflammation, or inflammation-associated disorder comprising a combination which includes an amount of a TNFα antagonist and an amount of a cyclooxygenase-2 selective inhibitor or prodrug thereof which together constitute a pain or inflammation suppressing treatment or prevention effective amount of the combination.

49. A pharmaceutical composition comprising a pharmaceutically-acceptable excipient and a combination which includes, an amount of a TNFα antagonist and an amount of a cyclooxygenase-2 selective inhibitor or prodrug thereof which together constitute a pain or inflammation suppressing treatment or prevention effective amount of the combination.

50. A kit that is suitable for use in the treatment, prevention or inhibition of pain, inflammation or inflammation-associated disorder, the kit comprises a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

51. A method for the treatment, prevention, or inhibition of a cardiovascular disease or disorder in a subject in need of such treatment, prevention, or inhibition, the method comprising administering to the subject a cyclooxygenase-2 selective inhibitor or prodrug thereof and a TNFα antagonist

52. The method according to claim 51, wherein the method comprises administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of a cardiovascular disease or disorder.

53. The method according to claim 51, wherein the TNFα antagonist comprises a compound that is selected from the group consisting of etanercept, infliximab, anti-TNFα, D2E7 human Mab, CDP-870, humicade, PEGylated soluble TNFα Receptor-1, TBP-1, PASSTNF-alpha®, AGT-1, ienercept, CytoTAb®, TACE, small molecule TNF mRNA synthesis inhibitor, PEGylated p75 TNFR Fc mutein, and TNFα antisense inhibitor.

54. The method according to claim 53, wherein the cyclooxygenase-2 selective inhibitor is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, meloxicam, prodrugs of any of them, and mixtures thereof.

55. A composition for the treatment, prevention, or inhibition of cardiovascular disease or disorder comprising a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

56. A pharmaceutical composition for the treatment, prevention, or inhibition of cardiovascular disease or disorder comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of a cardiovascular disease or disorder.

57. A kit that is suitable for use in the treatment, prevention or inhibition of a cardiovascular disease or disorder, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of a cardiovascular disease or disorder.

58. A method for the treatment, prevention, or inhibition of cancer in a subject in need of such treatment, prevention, or inhibition, the method comprising administering to the subject a cyclooxygenase-2 selective inhibitor or prodrug thereof and a TNFα antagonist which is selected from the group consisting of a compound that affects the synthesis of TNFα, a compound that inhibits the binding of TNFα with a receptor specific for TNFα, and a compound that interferes with intracellular signaling triggered by TNFα binding with a receptor.

59. The method according to claim 58, wherein the method comprises administering to a subject that is in need of such prevention, treatment or inhibition a combination comprising an amount of a cycloxygenase-2 selective inhibitor or prodrug thereof and an amount of a TNFα antagonist wherein the amount of the cyclooxygenase-2 selective inhibitor and the amount of the TNFα antagonist together comprise an amount of the combination that is effective for the treatment, prevention, or inhibition of cancer.

60. The method according to claim 59, wherein the TNFα antagonist comprises a compound that is selected from the group consisting of etanercept, infliximab, anti-TNFα, D2E7 human Mab, CDP-870, humicade, PEGylated soluble TNFα. Receptor-1, TBP-1, PASSTNF-alpha®, AGT-1, ienercept, CytoTAb®, TACE, small molecule TNF mRNA synthesis inhibitor, PEGylated p75 TNFR Fc mutein, and TNFα antisense inhibitor.

61. The method according to claim 60, wherein the cyclooxygenase-2 selective inhibitor is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, meloxicam, prodrugs of any of them, and mixtures thereof.

62. A composition for the treatment, prevention, or inhibition of cancer comprising a combination which includes an amount of a TNFα antagonist and an amount of a cyclooxygenase-2 selective inhibitor or prodrug thereof which together constitute an amount of the combination that is effective for the treatment, prevention, or inhibition of cancer.

63. A pharmaceutical composition for the treatment, prevention, or inhibition of cancer comprising a pharmaceutically-acceptable excipient and a combination which includes an amount of a TNFα antagonist and an amount of a cyclooxygenase-2 selective inhibitor or prodrug thereof which together constitute an amount of the combination that is effective for the treatment, prevention, or inhibition of cancer.

64. A kit that is suitable for use in the treatment, prevention or inhibition of cancer, the kit comprising a first dosage form comprising a TNFα antagonist and a second dosage form comprising a cyclooxygenase-2 selective inhibitor or prodrug thereof, in quantities which comprise a therapeutically effective amount of the combination of the compounds for the treatment, prevention, or inhibition of cancer.

65. The method according to claim 1, wherein the method comprises administering to the subject a combination comprising:
a cyclooxygenase-2 selective inhibitor which is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
a TNFα antagonist which comprises an antibody molecule having specificity for human TNFα, comprising the light chain variable region having the sequence given in SEQ ID NO:4 and the heavy chain variable region having the sequence given in SEQ ID NO:2.

66. The method according to claim 1, wherein the method comprises administering to the subject a combination comprising:
a cyclooxygenase-2 selective inhibitor which is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
an antibody molecule having specificity for human TNFα, where the antibody molecule is a modified Fab fragment comprising a heavy chain having the sequence given in SEQ ID NO:6 and a light chain having the sequence given in SEQ ID NO:4.

67. The method according to claim 1, wherein the method comprises administering to the subject a combination comprising:
a cyclooxygenase-2 selective inhibitor which is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a lysys-maleimide group wherein each amino group of the lysyl residue has covalently linked to it a poly(ethyleneglycol) or methoxypoly(ethyleneglycol) polymer.

68. A therapeutic composition comprising an amount of a TNFα antagonist and an amount of a cyclooxygenase-2 selective inhibitor or prodrug thereof which together constitute a therapeutic amount of the combination.

69. The therapeutic composition according to claim 68, comprising:
a cyclooxygenase-2 selective inhibitor which is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
a TNFα antagonist which comprises an antibody molecule having specificity for human TNFα, comprising the light chain variable region having the sequence given in SEQ ID NO:4 and the heavy chain variable region having the sequence given in SEQ ID NO:2.

70. The therapeutic composition according to claim 68, comprising:
a cyclooxygenase-2 selective inhibitor which is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
an antibody molecule having specificity for human TNFα, where the antibody molecule is a modified Fab fragment comprising a heavy chain having the sequence given in SEQ ID NO:6 and a light chain having the sequence given in SEQ ID NO:4.

71. The therapeutic composition according to claim 68, comprising:
a cyclooxygenase-2 selective inhibitor which is selected from the group consisting of celecoxib, COX 189, JTE-522, deracoxib, a chromene, a chroman, parecoxib, valdecoxib, etoricoxib, rofecoxib, N-(2-cyclohexyloxynitrophenyl)methane sulfonamide, ABT963, BMS347070, meloxicam, prodrugs of any of them, and mixtures thereof; and
a compound comprising an antibody molecule having specificity for human TNFα, wherein said antibody molecule is a modified Fab fragment comprising a heavy chain having the sequence given in SEQ ID NO:6 and a light chain having the sequence given in SEQ ID NO:4, and having attached to one of the cysteine residues at the C-terminal end of the heavy chain a poly(ethyleneglycol) or methoxypoly(ethyleneglycol) polymer.

72. The therapeutic composition according to claim 68, comprising:
a cyclooxygenase-2 selective inhibitor which is selected from the group consisting of celecoxib, COX 189, parecoxib, valdecoxib, etoricoxib, rofecoxib, ABT963, BMS347070, meloxicam, prodrugs of any of them, and mixtures thereof; and
an antibody to human TNFα, selected from the group consisting of CDP-870, D2E7, ENBREL®, and REMICADE®.

73. The therapeutic composition according to claim 68, comprising CDP-870 and celecoxib.

74. The method according to claim 1, comprising administering to the subject a cyclooxygenase-2 selective inhibitor which is selected from the group consisting of celecoxib, parecoxib, valdecoxib, etoricoxib, rofecoxib, ABT963, BMS347070, COX 189, meloxicam, prodrugs of any of them, and mixtures thereof; and
an antibody to human TNFα, selected from the group consisting of CDP-870, D2E7, ENBREL®, and REMICADE®.

75. The method according to claim 1, comprising administering to the subject CDP-870 and celecoxib.


SEQUENCE LISTING (6 sequences)

SEQ ID NO: 1 - DNA, 648 bp, Artificial sequence
  Grafted Heavy Chain for Fab
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagtt

SEQ ID NO: 2 - protein, 216 aa, Artificial sequence
  Grafted Heavy Chain for Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 3 - DNA, 642 bp, Artificial sequence
  Grafted Light Chain for Fab and Modified Fab
gacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaagccctcatctacagtgcctctttcctctatagtggtgtaccatacaggttcagcggatccggtagtggtactgatttcaccctcacgatcagtagcctccagccagaagatttcgccacttattactgtcaacagtataacatctacccactcacattcggtcagggtactaaagtagaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagctttaatagaggagagtgt

SEQ ID NO: 4 - protein, 214 aa, Artificial sequence
  Grafted Light Chain for Fab and Modified Fab
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 687 bp, Artificial sequence
  Grafted Heavy Chain for Modified Fab
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 6 - protein, 229 aa, Artificial sequence
  Grafted Heavy Chain for Modified Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA